(19) United States
(12) Patent Application Publication
(10) Pub. No.: US 2023/0399701 A1
(43) Pub. Date: Dec. 14, 2023

(54) PROGNOSTIC GENE SIGNATURE AND METHOD FOR DIFFUSE LARGE B-CELL LYMPHOMA PROGNOSIS AND TREATMENT

(71) Applicant: The Children’s Mercy Hospital, Kansas City, MO (US)

(72) Inventors: Todd Christopher Bradley, Kansas City, MO (US); Santosh Khanal, Kansas City, MO (US)

(21) Appl. No.: 18/250,899

(22) PCT Filed: Oct. 27, 2021
(86) PCT No.: PCT/US2021/056774

§ 371 Date: Apr. 27, 2023

Related U.S. Application Data
(60) Provisional application No. 63/105,970, filed on Oct. 27, 2020.

(51) Int. Cl. C12Q 1/6886

(52) U.S. Cl. CPC C12Q 1/6886; C12Q 2600/118; C12Q 2600/158; C12Q 2600/106

(57) ABSTRACT

Systems, treatment and prognostic methods, and kits for risk stratification and development of treatment options for diffuse large B-cell lymphoma patients. The systems, methods, and kits comprise determining, detecting, and evaluating gene expression values for at least ALDOC, ASIP, ATP8A1, CD IE, DUSP16, FAF1, FAM223A1FAM223B, GAREM, GNG8, LM02, LPPR4, LY75, NMAEL, PAD 12, PDK1, PPP1R7, SCN1A, SLAMF1, SSTR2, TNFRSF9, USH2A, VEZF1, WDR91, ADRA2B, ECT2, ELOVL6, IGSF9, NEK3, PDK4, PES1, PUSL1, TAD A2A, and ZMYND19, or a subset thereof, detected in a biological sample from the patient and determining a risk score associated with the gene signature panel, which can be used to guide treatment of the patient.

### CROSS-REFERENCE TO RELATED APPLICATIONS

The present application claims the priority benefit of U.S. Provisional Patent Application Ser. No. 63/105,970, filed Oct. 27, 2020, entitled PROGNOSTIC GENE SIGNATURE AND METHOD FOR DIFFUSE LARGE B-CELL LYMPHOMA PROGNOSIS AND TREATMENT, incorporated by reference in its entirety herein.

### BACKGROUND OF THE DISCLOSURE

### Field of the Invention

The present invention relates to a prognostic gene panel and methods and systems of using the gene signature to risk stratify and treat certain types of cancer patients.

### Description of Related Art

Diffuse large B-cell lymphoma (DLBCL) is the most common type of non-Hodgkin lymphoma and can have variable response to therapy and long-term clinical outcomes. DLBCL is of B-cell origin and was typically treated with a regimen of cyclophosphamide, hydroxydaunorubicin, oncovin and prednisone (CHOP) but the addition of the anti-CD20 monoclonal antibody rituximab (R) significantly improved patient overall-survival outcomes. R-CHOP is now regarded as the superior treatment strategy and represents the current standard of care for most DLBCL, though investigation in more other targeted therapies is underway.

A scoring system was developed to identify risk groups of DLBCL individuals called the International Prognostic Index (IPI) that uses age, lactate dehydrogenase levels, general health status, stage of tumor and number of disease sites to place the patients in 1 of 4 risk groups that correspond with the likelihood of 3-year overall survival (see International Non-Hodgkin's Lymphoma Prognostic Factors, A predictive model for aggressive non-Hodgkin's lymphoma. N Engl J Med 329, 987-994 (1993)). The IPI was largely developed based on studies of patients before immunotherapy was widely used as a treatment strategy. A revised IPI (R-IPI) using R-CHOP-treated patients was developed that had improved prognostic value at determining risk groups. (see Sehn et al. The revised International Prognostic Index (R-IPI) is a better predictor of outcome than the standard IPI for patients with diffuse large B-cell lymphoma treated with R-CHOP. Blood 109, 1857-1861 (2007)). This metric provides discrete prognostic values that inform treatment strategies and clinical follow-up. For R-IPI scoring, a score of 0 is classified as “very good,” a score of 1 or 2 is classified as “good,” while a score of 3, 4 or 5 is classified as “poor.”

Gene expression profiling studies of DLBCL have reported at least two histologically indistinguishable subclasses of DLBCL based on gene expression of approximately 90 genes; the germinal center B-cell-like (GCB) and the activated B-cell-like (ABC). In addition to subclass identity, it was indicated that overall survival time was significantly higher in the GCB subclass than in those with ABC subclass of DLBCL. Moreover, the two subclasses also differ in clinical presentation and response to therapy. Another study identified a molecular subclass of DLBCL that was distinct from GCB or ABC and was termed type3 and identified a 17 gene signature that could predict overall survival after therapy. This led to further prospective studies that proposed prognostic gene signatures consisting of 6, 7, 13, 14 or 108 genes.

Despite the identification of various prognostic gene sets, there are many challenges that have impeded their clinical implementation; (i) the lack of reproducibility in various datasets, (ii) the lack of overlap of genes in the different signatures, (iii) technologies utilized to generate gene expression values (e.g., Microarray vs RNA-sequencing), and (iv) the effect of newer therapies such as the addition of rituximab to therapy on survival outcomes.

### SUMMARY OF THE DISCLOSURE

To address these deficiencies in current clinical information, gene expression and clinical parameters in the Lymphoma/Leukemia Molecular Profiling Project from individuals that received R-CHOP therapy were used to identify genes whose expression is associated with overall survival and further refined this to develop a prognostic gene signature of 33 genes that could be used to calculate risk scores for each individual and predict overall survival. Moreover, we validated this prognostic gene signature in 3 additional data sets and determined significant differences in overall survival in individuals with high or low risk scores. The prognostic gene signature could identify individuals at high-risk for poor outcomes after traditional DLBCL diagnosis and treatment, and support use of newer experimental therapies for such patients.

In one aspect, there are provided methods for diffuse large B-cell lymphoma prognosis and treatment in a patient in need thereof. The methods generally comprise determining a first gene expression profile in a biological sample from the patient for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91; and correlating increased expression levels of the genes with improvement in overall survival outcomes in the patient. The method further comprises determining a second gene expression profile in the biological sample for at least a second set of genes ADRA2B, ECT2, ELOVL6, IGSF9, NEK3, PDK4, PES1, PUSL1, TADA2A, and ZMYND19; and correlating low expression levels of the second set of genes with improvement in overall survival outcomes in the patient. In one aspect, there are provided methods of treating diffuse large B-cell lymphoma in a patient in need thereof. The methods generally comprise receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, WDR91, ADRA2B, ECT2, ELOVL6, IGSF9, NEK3, PDK4, PES1, PUSL1, TADA2A, and ZMYND19, or subset thereof, detected in a biological sample from the patient;

determining a risk score for the patient based upon increased or decreased expression of each gene expression value as compared to a reference standard; and administering a therapeutic agent to the patient to treat the diffuse large B-cell lymphoma. Preferably, the therapeutic agent comprises a standard of care active agent (e.g., R-CHOP) when the risk score is low. Conversely, the therapeutic agent comprises an adjunctive chemotherapeutic, experimental therapy, and/or aggressive active agent against the diffuse large B-cell lymphoma when the risk score is high.

Also described herein are systems for diffuse large B-cell lymphoma prognosis and treatment in a patient in need thereof. The systems generally comprise a user interface for receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZFl, and WDR91 in a biological sample from the patient to generate a first gene expression profile; computer readable memory to store the first gene expression profile; at least one database comprising a reference standard for each of the first set of genes; a processor with a computer-readable program code comprising instructions for comparing the first gene expression profile with the reference standard data correlating increased expression levels of the first set of genes with improvement in overall survival outcomes in the patient, and calculating a risk score; and an output for reporting a risk score for the patient.

In one aspect, methods are also disclosed for diffuse large B-cell lymphoma prognosis and treatment in a patient in need thereof. The methods generally comprise receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF 1, and WDR91 in a biological sample from the patient; generating a first gene expression profile; comparing the first gene expression profile with a reference standard data for each of the genes; correlating increased expression levels of the first set of genes with improvement in overall survival outcomes in the patient; and calculating a risk score predictive of overall survival for the patient. The methods can further comprise receiving gene expression values for at least ADRA2B, ECT2, ELOVL6, IGSF9, NEK3, PDK4, PES1, PUSL1, TADA2A, and ZMYND19 in the biological sample from the patient; generating a second gene expression profile; and likewise calculating a risk score predictive of overall survival for the patient based upon the combined information.

The present disclosure also concerns kits for diffuse large B-cell lymphoma prognosis and treatment in a patient in need thereof. The kits generally comprise a plurality of probes each having binding specificity for a target gene in a gene panel comprising ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF 1, WDR91, ADRA2B, ECT2, ELOVL6, IGSF9, NEK3, PDK4, PES1, PUSL1, TADA2A, and ZMYND19, or a gene product thereof; optional reagents and/or buffers; and instructions for mixing the probes with a biological sample obtained from the patient. Instructions can also be included for sample preparation and handling.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph showing the median expression of two genes that when highly expressed are significantly associated with favorable (SSTR2) or unfavorable (IGSF9) 5-year OS in R-CHOP treated DLBCL displayed as a Kaplan-Meier plot for OS of the high and low expression groups of individuals. P value is the result of a log-rank test.

FIG. 1B is a heatmap of the z-scores based on gene expression of the 33 genes that are a part of the prognostic gene signature associated with OS grouped by individuals with high and low risk scores.

FIG. 1C is a Kaplan-Meier plot of DLBCL OS when individuals are grouped into high and low risk groups. P values shown are a result of a log-rank test.

FIG. 1D is a Kaplan-Meier plot of DLBCL OS when individuals are grouped into risk groups based on quartiles of risk score with the lowest quartile (Q1), second (Q2), third (Q3) and highest (Q4). P values shown are a result of a log-rank test.

FIG. 1E is an illustration of the top significantly enriched molecular pathways determined by Metascape shown as a network of enriched terms grouped by cluster.

FIG. 2A demonstrates that the prognostic gene signature can predict survival independent of R-IPI. A graph of a Kaplan-Meier plot of DLBCL OS when individuals are grouped into high and low risk groups using R-IPI scores. P values shown are a result of a log-rank test.

FIG. 2B shows a bar graph showing the frequency of R-IPI scores for individuals in low or high risk score groups based on prognostic gene signature expression.

FIG. 2C shows Kaplan-Meier plots of DLBCL OS when individuals are grouped into high and low risk groups using risk scores developed using only samples with low R-IPI scores (0-1; n=71; left) or intermediate R-IPI scores (2-3; n=78; right). P values shown are a result of a log-rank test.

FIG. 3A is a graph showing the analysis of the prognostic gene signature within DLBCL subtypes. Shows a Kaplan-Meier plot of DLBCL OS when individuals are grouped into high and low risk groups using risk scores determined from the full dataset using only samples with the DLBCL molecular subtype of germinal center B cell (GCB). P values shown are a result of a log-rank test.

FIG. 3B is the same analysis as in FIG. 3A, except using risk scores determined from the full dataset using only samples with the DLBCL molecular subtype of activated B cell (ABC). P values shown are a result of a log-rank test.

FIG. 3C is a Kaplan-Meier plot of DLBCL OS when individuals are grouped into high and low risk groups using risk scores developed using only samples with the DLBCL molecular subtype of GCB. P values shown are a result of a log-rank test.

FIG. 3D is a Kaplan-Meier plot of DLBCL OS when individuals are grouped into high and low risk groups using risk scores developed using only samples with the DLBCL molecular subtype of ABC. P values shown are a result of a log-rank test.

FIG. 4. shows data from validation of the prognostic gene signature in external DLBCL datasets. Kaplan-Meier plots of DLBCL OS are shown when individuals are grouped into high and low risk groups using risk scores determined from the LLMPP dataset using 3 external DLBCL datasets (GSE34171, GSE32918/69051 and TCGA). P values shown are a result of a log-rank test.

FIG. 5 is a logic flow diagram illustrating an exemplary process for assessing risk values using the genomic risk scoring system, optionally in combination with the established R-IPI scoring system.

FIG. 6 is a graph of LASSO coefficient analysis on 61 features. 33 marker genes were selected using 10-fold cross-validation with the minimum value of log (□□-3.3 based on the 1 standard error criteria. The C-index (concordance index) on the y-axis is a measure of the goodness of fit in the model. The region between vertical dashed lines represents models within one standard error of the minimum, which is the most regularized form, for the selected C-index value.

### DETAILED DESCRIPTION

The present invention is concerned with a unique molecular prognostic signature that is useful for predicting DLBCL prognosis, regardless of subtype. In particular, the present invention relates to methods and reagents for detecting and profiling the expression levels of combinations 10 of these genes, and methods of using the detected expression levels in calculating a clinical outcome or risk score for DLBCL patients, regardless of subtype. As used here, the “expression level” or similar phrases refer to the level of expression of gene products from the target genes, which can be indicated by the amount of RNA transcripts or proteins detected, the quantity of DNA detected, detected enzymatic activities, and the like depending upon the type of detection technique and substrates or probes used for detection.

The methods involve detection of expression levels of genes from a biological sample obtained from a DLBCL patient. Biological samples include liquid or tissue samples obtained from the patient, such as liquid or solid tumor tissue biopsies, lymph node biopsies, bone marrow aspirate, blood, serum, and the like. Depending upon the assay kit or system used, the sample is processed and then analyzed to detect expression levels of the target genes. Sample processing includes diluting and/or enriching the sample, e.g., with suitable buffers and/or reagents, and assaying the sample in accordance with the selected approach. Numerous commercially-available kits and/or services are available for detection of expression levels of genes or gene products, including associated software for generating a gene expression value for each target gene (or product) detected in the sample. These gene expression values can then be analyzed using the prognostic gene panel described herein to determine the patient's risk profile.

The expression levels of the genes in combination indicate an increased risk of an unfavorable clinical outcome (without further treatment intervention) or improved survival outcomes depending upon the detected expression level of the particular genes. In one or more embodiments, the prognostic gene panel can be used to predict a risk score for a DLBCL patient, and in particular predict a successful or unsuccessful outcome from the current therapeutic standard of care. Thus, the term “prognosis” and variations thereof are used herein to refer to a predicted clinical outcome, such as likelihood of high overall survival (e.g., without relapse or progression for a period of time) or low overall survival associated with DLBCL, such as relapse or progression (e.g., metastasis), etc. which prediction is based upon the expression level of the combinations of genes disclosed herein. The term “prediction” and variations thereof are used herein to refer to the likelihood that a patient will have a favorable or unfavorable survival outcome, and in one or more embodiments, whether the patient will respond either favorably or unfavorably to the current standard of care (e.g., R-CHOP).

Thus, the 33-gene molecular prognostic signature or subset thereof can be used to identify patients for which alternative, adjunctive, and/or experimental therapies should be considered earlier in the treatment protocol. In one or more embodiments, the 33-gene molecular prognostic signature or subset thereof can be used to identify patients for which earlier intervention or aggressive treatment may be recommended. In one or more embodiments, the 33-gene molecular prognostic signature or subset thereof can be used to risk stratify patients for more aggressive treatment considerations. In one or more embodiments, the 33-gene molecular prognostic signature or subset thereof can be used to design and select patients for a clinical trial. In one or more embodiments, the 33-gene molecular prognostic signature or subset thereof can be used to analyze the outcome of a clinical trial and further analyze success or failure of the treatments explored therein.

In one or more embodiments, the 33-gene molecular prognostic signature or subset thereof can also be used to monitor treatment efficacy, such as by comparing patient expression levels before and after a given treatment. The 33-gene molecular prognostic signature or subset thereof can also be used overtime to provide an indication of disease progression and/or response to treatment.

TABLE 1
Multivariate DLBCL prognostic gene signature - 33 gene panel.

| | | | Coeffi- | Hazard | |
|---|---|---|---|---|---|
| Gene | Log-rank | Hazard | cient | ratio | Lasso |
| name | P value | ratio | beta | pvalue | coefficient |
| ADRA2B | 0.00053225 | 2.5 | 0.93 | 0.00083 | 0.05929974 |
| ALDOC | 6.26E−06 | 0.28 | −1.3 | 2.30E−05 | −0.2266974 |
| ASIP | 0.00055649 | 0.4 | −0.93 | 0.00085 | −0.0994086 |
| ATP8A1 | 2.06E−05 | 0.31 | −1.2 | 5.70E−05 | −0.052468 |
| CD1E | 0.00020092 | 0.37 | −1 | 0.00036 | −0.1111254 |
| DUSP16 | 0.00053301 | 0.39 | −0.93 | 0.00083 | −0.0963421 |
| ECT2 | 0.00062699 | 2.5 | 0.92 | 0.00095 | 0.13182723 |
| ELOVL6 | 0.00083533 | 2.5 | 0.9 | 0.0012 | 0.055146 |
| FAF1 | 0.00044069 | 0.38 | −0.96 | 0.00071 | −0.0652772 |
| FAM223A\| | 0.00017197 | 0.36 | −1 | 0.00032 | −0.0121265 |
| FAM223B | | | | | |
| GAREM | 0.00091943 | 0.41 | −0.89 | 0.0013 | −0.0299263 |
| GNG8 | 0.0004221 | 0.38 | −0.96 | 0.00069 | −0.0089058 |
| IGSF9 | 9.19E−06 | 3.4 | 1.2 | 3.00E−05 | 0.19446142 |
| LMO2 | 0.00023192 | 0.37 | −1 | 0.00041 | −0.0070721 |
| LPPR4 | 0.00085777 | 0.41 | −0.9 | 0.0013 | −0.1433395 |
| LY75 | 9.00E−05 | 0.35 | −1.1 | 0.00018 | −0.252489 |
| MAEL | 0.00014479 | 0.35 | −1 | 0.00028 | −0.086909 |
| NEK3 | 0.000653 | 2.5 | 0.9 | 0.00098 | 0.08073014 |
| PADI2 | 0.0002852 | 0.37 | −0.98 | 0.00049 | −0.0332634 |
| PDK1 | 0.00094706 | 0.41 | −0.89 | 0.0014 | −0.0435511 |
| PDK4 | 0.0001327 | 2.8 | 1 | 0.00025 | 0.18311325 |
| PES1 | 0.00080774 | 2.4 | 0.89 | 0.0012 | 0.09271489 |
| PPP1R7 | 0.00060029 | 0.39 | −0.93 | 0.00093 | −0.2483229 |
| PUSL1 | 0.00013295 | 2.8 | 1 | 0.00025 | 0.14247471 |
| SCNIA | 0.00059538 | 0.39 | −0.93 | 0.00093 | −0.054923 |
| SLAMF1 | 0.00049663 | 0.39 | −0.93 | 0.00078 | −0.0094785 |
| SSTR2 | 2.65E−06 | 0.27 | −1.3 | 1.20E−05 | −0.0260066 |
| TADA2A | 0.00010716 | 2.8 | 1 | 0.00021 | 0.12055065 |
| TNFRSF9 | 0.00094243 | 0.41 | −0.88 | 0.0014 | −0.004922 |
| USH2A | 0.00012899 | 0.35 | −1 | 0.00025 | −0.1920536 |
| VEZF1 | 0.00021363 | 0.37 | −1 | 0.00038 | −0.3893348 |
| WDR91 | 0.000353 | 0.38 | −0.97 | 0.00059 | −0.0041198 |
| ZMYND19 | 0.00089279 | 2.4 | 0.88 | 0.0013 | 0.26520514 |

In one or more embodiments, the method comprises detecting the expression level of at least ADRA2B (Adrenoceptor Alpha 2B), ALDOC (Aldolase, Fructose-Bisphosphate C), ASIP (Agouti Signaling Protein), ATP8A1 (ATPase Phospholipid Transporting 8A1), CD 1E (CD1e Molecule), DUSP16 (Dual Specificity Phosphatase 16), ECT2 (Epithelial Cell Transforming 2), ELOVL6 (ELOVL Fatty Acid Elongase 6), FAF1 (Fas Associated Factor 1), FAM223A1FAM223B (Family With Sequence Similarity 223 Member AlFamily With Sequence Similarity 223 Member B), GAREM (GRB2 Associated Regulator of MAPK1), GNG8 (G Protein Subunit Gamma 8), IGSF9 (Immunoglobulin Superfamily Member 9), LMO2 (LEVI Domain Only 2), LPPR4 (Lipid Phosphate Phosphatase-Related Protein type 4), LY75 (Lymphocyte Antigen 75), MAEL (Maelstrom Spermatogenic Transposon Silencer), NEK3 (NIMA Related Kinase 3), PADI2 (Peptidyl Arginine Deiminase 2), PDK1 (Pyruvate Dehydrogenase Kinase 1), PDK4 (Pyruvate Dehydrogenase Kinase 4), PES1 (Pescadillo Ribosomal Biogenesis Factor 1), PPP1R7 (Protein Phosphatase 1 Regulatory Subunit 7), PUSL1 (Pseudouridine Synthase Like 1), SCN1A (Sodium Voltage-Gated Channel Alpha Subunit 1), SLAWIF1 (Signaling Lymphocytic Activation Molecule Family Member 1), SSTR2 (Somatostatin Receptor 2), TADA2A (Transcriptional Adaptor 2A), TNFRSF9 (TNF Receptor Superfamily Member 9), USH2A (Usherin), VEZF1 (Vascular Endothelial Zinc Finger 1), WDR91 (WD Repeat Domain 91), and/or ZMYND19 (Zinc Finger MYND-Type Containing 19), or a subset thereof.

In one or more embodiments, the method comprises detecting the expression level of at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91 in the patient, and correlating increased expression levels of the genes with improvement in overall survival outcomes in the patient (i.e., a low risk score). In other words, high expression levels of these genes (particularly SSTR2) are correlated with higher overall survival and low expression levels of the genes are correlated with lower overall survival outcomes in the patient. Thus, the expression levels of these particular genes are directly correlated to positive survival outcomes.

In one or more embodiments, the method comprises detecting the expression level of at least ADRA2B, ECT2, ELOVL6, IGSF9, NEK3, PDK4, PES1, PUSL1, TADA2A, and ZMYND19 in the patient, and correlating low expression levels of the genes with improvement in overall survival outcomes in the patient. In other words, increased expression levels of the genes (particularly IGSF9) are correlated with lower survival outcomes (i.e., a high risk score), whereas low expression levels are correlated with higher survival outcomes. Thus, the expression levels of these genes are inversely correlated to positive survival outcomes.

As used herein, low or lower survival outcomes or overall survival refers to an increased risk (high or higher risk) of death due to DLBCL as compared to DLBCL patients (with the same subtype if applicable) having a higher survival outcome or overall survival (low or lower risk of death). A higher risk score denotes a higher mortality risk for individuals with DLBCL. In the DLBCL field, a 3-year overall survival window is often the benchmark for gauging risk. In one or more embodiments, the inventive prognostic signature panel can be used to predict individuals with higher or lower risk over a 5-year overall survival window.

Risk score stratification is carried out by first assessing the median risk score of a population, e.g., based upon gene expression profiling, to develop the reference standard (e.g., median expression value). Profiling data can be obtained from within the study being carried out or can be from publicly accessible data, such as from the Gene Expression Omnibus. In one or more embodiments, a “low” risk score is a score below the median risk score using the innovative panel and analysis. In one or more embodiments, a “high” risk score is a score above the median risk score using the innovative panel and analysis. Unlike R-IPI, the risk scores here are not static values. Rather, the actual values will differ depending on the type of technology used to calculate gene expression (e.g., microarray vs. RNA-sequencing). For example, in the population studied, using microarray analysis via the Affymetrix Human Genome U133 Plus 2.0 Array, the median value was −8.422649568. Thus, a “low risk” score would be assigned to any scores falling below the median value, and a “high risk” score would be assigned to any scores falling above the median value. Approaches for calculating gene expression values using the different technologies are known in the art.

In one or more embodiments, the method comprises detecting the expression level of a combination of the foregoing target genes in a biological sample obtained from the patient and correlating their expression levels with either increased or decreased overall survival, as noted. The combined information yields a risk score that can be used to risk stratify the patient and inform treatment decisions.

In one or more embodiments, the method comprises detecting the expression level of all 33 genes in the panel listed in Table 1. In one or more embodiments, the biological sample is screened for expression levels of the panel of 33 genes in Table 1. In one or more embodiments, the gene expression level data is provided or received for analysis. In other words, the gene expression levels have already been detected and/or determined, such as in a separate study or analysis or by a different laboratory or practitioner and provided for determination of a risk score. Thus, in one or more embodiments, the method itself involves receiving values corresponding to a patient's gene expression profile and screening the data and calculating a risk score based upon the gene expression levels. In one or more embodiments, the gene expression values are input by a user into a user interface, and compared against a reference standard for each gene to generate a risk score based upon the input values.

It will be appreciated that the biological sample can be screened and the gene expression levels can be detected and calculated various ways which have been established in the art. The expression level of the target genes can be determined by detecting, for example, various gene products, including RNA product of each target gene, such as mRNA transcripts, as well as proteins etc. Likewise, it will be appreciated that a number of techniques can be used to detect or quantify the level of gene products within a sample, including arrays, such as microarrays, RNA sequencing (e.g., PCR, including quantitative RT-PCR), next-generation sequencing (NGS), and the like. Illumina sequencing technology, sequencing by synthesis (SBS), is a widely adopted NGS technology. Various genotyping arrays and kits are commercially available and can include various reagents, e.g., for hybridization-based enrichment or PCR-based amplicon sequencing, as well as nucleic acid probes that are complementary or hybridizable to an expression product of the target genes. Quantitative expression levels of the target genes can also be determined via RT-PCR or quantitative PCR assays. Regarding proteins, it will be appreciated that various techniques can be used including immunoassays, such as Western Blot, ELISA, etc., which kits include antibodies having binding specificity for each of the target gene products. Nucleic acid or antibody fragments can also be used as probes, along with fluorescently-labeled derivatives thereof.

Commercially available kits for detecting gene expression levels often include associated software for generating a gene expression value. It will be appreciated that various approaches can be used to standardize or normalize expression values obtained from various techniques. For example, expression levels may be calculated by the A(ACt) method. Moreover, as further research is conducted, a calibrator or reference standard (control) can be developed for each gene as a point of comparison. Such reference standards or controls may be specific values or datasets associated with a particular survival outcome. In one embodiment, a dataset may be obtained from samples from a group of subjects known to have DLBCL and good survival outcome or known to have DLBCL and have poor survival outcome or known to have DLBCL and have benefited from a particular treatment or known to have DLBCL and not have benefited from a particular treatment. The expression data of the genes in the dataset can be used to create a control value that is used in testing new samples. In such an embodiment, the “control” or reference standard is a predetermined value or dataset for the 33 target genes or subset thereof. Control or reference standard values can also be obtained from healthy patients (without DLBCL) having “normal” levels of gene expression for each target gene. In such a case, “high” or “low” expression levels of the target genes can be compared against these normal values.

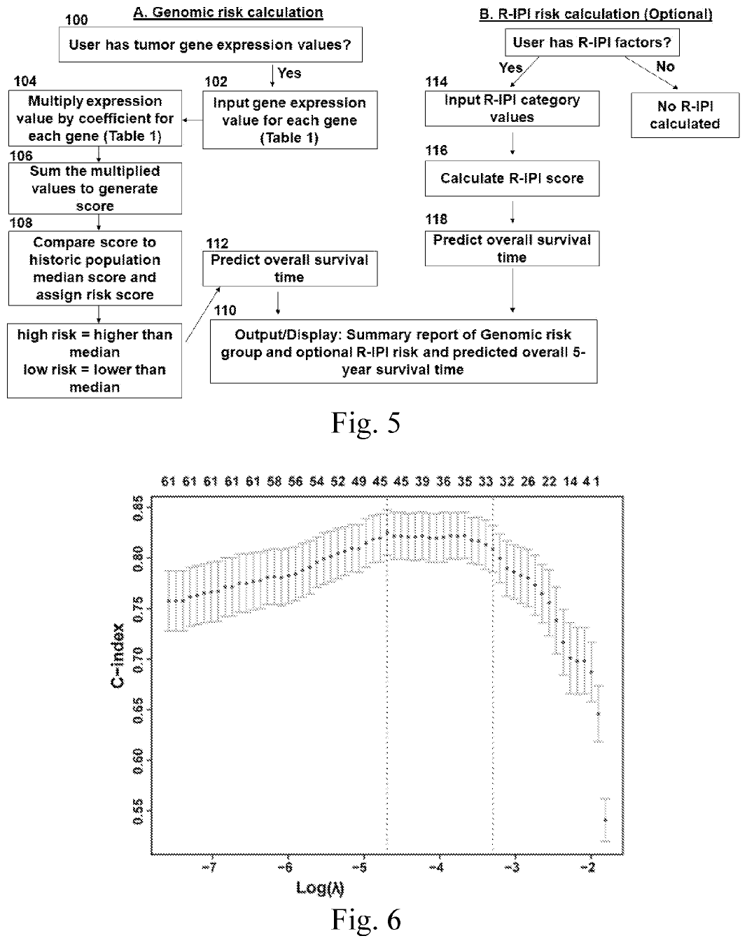

In one or more embodiments, with reference to FIG. 5, once the expression level (100) is determined or received/input (102), the total expression level of each gene is multiplied by its lasso coefficient noted in Table 1 (104), and the sum of the values are calculated to yield a risk score (106). Thus, the risk score is a measure of the summation of expression levels for the 33 genes (Table 1), each multiplied by a particular constant (e.g., lasso coefficient). It will be appreciated that this calculation may be carried out automatically using a computer implemented system and process for predicting a prognosis. The system can include a database comprising reference standards for each gene associated with a prognosis depending upon expression levels, such as historical median values (108). The system can further include a computer readable medium having stored thereon a data structure for storing the computer implemented risk score, as well as a database including records comprising reference standards for combinations of genes ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, WDR91, ADRA2B, ECT2, ELOVL6, IGSF9, NEK3, PDK4, PES1, PUSL1, TADA2A, and ZMYND19, or subset thereof. Additional components of the system can include a user interface capable of receiving gene expression values (102) for use in calculating the risk score and/or comparing to the reference standards in the database, as well as an output (110) which can display the risk score and/or the predicted prognosis of survival outcomes (112) for the patient. The output can also be used to inform treatment recommendations for the patient. In one or more embodiments, a web-based interface tool is provided for receiving gene expression values for use in calculating the risk score and/or comparing to the reference standards in the database, as well as an output which can display the risk score and/or the predicted prognosis of survival outcomes for the patient.

Methods herein can involve further analysis of the gene expression levels depending upon the DLBCL subtype of the patient, once known. For example, the methods can include detecting expression levels for at least CRCP, ZNF518A, SLC5Al2, TMEM37, EPOR1RGL3, LINC00917, CTB-43E15.1, ECT2, IGSF9, PLCB4, LINC005991MIR124-1, ING2, FAF1, ZNF236, AC091633.3, and USH2A in an ABC subtype DLBCL patient, and particularly IGSF9, ECT2, FAF1, USH2A, which overlap with the 33-gene prognostic signature above, and correlating expression levels to a risk score. The methods can include detecting expression levels for at least TNFRSF10A, CPT1A, ELOVL6, SNHG4, RP11-349E4.1, HAS3, LINC00933, CCDC126, CALML5, CD58, LOC339539, and SERTAD1 in a GCB subtype DLBCL patient, and particularly ELOVL6, which overlaps with the 33-gene prognostic signature above, and correlating expression levels to a risk score. These secondary risk scores can be used to further refine prognosis and inform treatment decisions when the subtype of the patient is known. Such secondary risk scores can also be used to establish and monitor risk over different time points as part of monitoring patient treatments and/or outcomes. Notably, however, the 33-gene panel in Table 1, has been shown to be accurate without regard to subtype.

It is envisioned that the novel 33-gene signature will be a useful tool for clinicians and researchers, and can be used alone or, with reference to FIG. 5, complementary to the IPI or R-IPI that is currently used to improve patient care. For example, patients having a low IPI score, which are determined to have a high risk profile by the novel gene signature described herein, should be more closely monitored and/or treated more aggressively than a patient receiving a low IPI and low risk score by the inventive gene signature. Likewise, a patient having a high IPI score and also a high risk profile using the inventive gene signature should be considered as candidates for earlier intervention, adjunctive therapies, more aggressive treatment protocols, and/or experimental therapies. Thus, the system, as illustrated in FIG. 5, can include the option of inputting known R-IPI factors for the patient (114) and calculating an R-IPI score (116) to provide additional details regarding the predicted survival (118) and display (110) the resulting risk score.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

In this study we have identified a prognostic gene signature that when calculated into a risk score could accurately predict survival time in individuals with DLBCL. When risk scores were calculated using this prognostic gene set in 3 additional published DLBCL study groups, individuals with low risk score had significantly better overall survival, indicating the robustness of the gene signature for multiple external datasets. This represents a significant improvement over previously identified prognostic gene signatures that are not reproducible across datasets or technologies.

Surprisingly, our prognostic signature gene panel has very little overlap with previously published prognostic gene lists for DLBCL (Table 3). Moreover, when we evaluated three of the previous prognostic gene signatures on the R-CHOP-treated LLMP DLBCL dataset where our gene signature was derived, only a fraction of the genes in each of the previous gene lists were individually associated with overall survival and could not individually predict overall survival as well as our newly-identified multivariate gene list. One gene, LA102, overlapped the 108 gene signature described to predict GCB DLBCL overall survival as well as two other studies to develop prognostic gene signatures. This gene has been shown to be over-expressed in normal germinal center B cells as well as B-cell lymphoma and may play a pivotal role in DLBCL pathogenesis as it reproducibly associates with OS in multiple studies.

It is encouraging that when using our gene signature in 4 independent studies, individuals with a high-risk score demonstrated significantly lower overall survival compared with individuals with low risk scores using our panel. Future studies of larger cohorts of DLBCL individuals with standardized treatment and biological factors (age, sex, ethnicity) and gene expression determined using a standardized technology such as Illumina sequencing will allow for benchmarking of all the prognostic gene signatures.

In addition to molecular profiling, the R-IPI is used in the clinic to determine prognosis in DLBCL. R-IPI is a revised standard incorporating the characteristics of rituximab immunotherapy. It uses the parameters of age, ECOG performance status, lactase dehydrogenase levels, number of extranodal tumor sites, and tumor stage to develop a score (Sehn et al., 2007). It is a critical index that guides treatment decisions and clinical trial enrollment. When we developed risk scores using our identified prognostic gene signature, individuals with high risk had significant lower overall survival even in individuals with low or intermediate R-IPI scores. This demonstrates that our prognostic gene signature could improve survival prediction over the R-IPI, alone, and could be used in conjunction with the R-IPI to improve clinical decision making.

Other genetic predictors are also being used in addition to molecular profiling and clinical parameters, which contribute to the understanding of the mechanisms of DLBCL pathogenesis and predicting survival. For example, using specific genetic alterations, driver mutations and copy number to group DLBCL into subtypes has been shown to predict outcome, but also provide a temporal landscape of DLBCL progression . The potential of combining genetic alteration, gene expression profiling and other indexes such as R-IPI will result in the most accurate classification of individuals with DLBCL in order to predict overall survival and risk.

Enrichment of cellular pathways were restricted to thioester metabolism and hormone signaling through GPCR and generally were involved in metabolism. Many of the individual genes on the list have previously been associated with lymphoma; DUSP16 controls MAPK signaling, SLAMF1 which encodes CD150 and TNFRSF9 which encodes 4-1BB and have been shown to play a role in lymphocyte regulation and growth. Moreover, LY75, that encodes CD205, is an active target for therapeutic antibody generation in non-Hodgkin's lymphoma. Thus, further exploration of the individual genes in our prognostic gene signature may identify new therapeutic targets for DLBCL.

Our gene signature can predict survival based on low and high-risk individuals in multiple published datasets that utilized different technologies to determine tumor gene expression. The absolute value of the risk scores were variable between the datasets. This could be because differences in the individuals within the cohorts or differences in the methods used to generate the gene expression values (e.g., Microarray vs. RNA-seq). For prospective assignment of DLBCL patients to high or low risk, the technology used to generate the gene expression values needs to be considered or further efforts to standardize these gene values across platforms will be required. Since Illumina RNA-seq is becoming a standard for transcriptome sequencing, perhaps the absolute risk scores identified in the TCGA dataset are the most relevant for prospective risk phenotyping, with the caveat of having a small number of DLBCL patients to date. Future studies using RNA-seq from larger cohorts of individuals with DLBCL can help determine if RNA-seq is the optimal technology to determine risk scores in the clinical setting for individual DLBCL patients.

As new therapies for lymphoma become available, including new immunotherapies and personalized medicine approaches such as CAR-T cells it will be important to identify candidate individuals that are at high-risk and may benefit from experimental therapeutic approaches compared with individuals that will have lower-risk of death with current therapies. Focusing on the high-risk individuals that have a lower OS may require a different therapeutic approach and identify novel targets for therapy. The addition of our prognostic gene signature to IPI, and other clinical parameters, may provide clinicians and patients with one more tool in the toolbox to better guide therapeutic decisions in patients with DLBCL.

### METHODS

Datasets Used in this Study and Data Availability

We used gene expression and clinical results from 233 clinical DLBCL samples from individuals that underwent R-CHOP therapy that was previously published with the data available in GEO (Gene Expression Omnibus) under the accession number GSE10846. In these previous studies, samples were taken from lymph node tissue of each patient. Total RNA was extracted using All Prep RNA/DNA kit (Qiagen, Valencia, Calif.) according to the manufacturers' protocols. Biotinylated cRNA were prepared according to the standard Affymetrix protocol from 1 microg mRNA (Expression Analysis Technical Manual, 2001, Affymetrix). Following fragmentation, 11 micrograms of cRNA were hybridized for 16 hours at 45 C. on U133 plus 2.0 arrays from Affymetrix. Arrays were washed and stained in the Affymetrix Fluidics Station 400. Scanning was performed by the Affymetrix 3000 Scanner. The data were analyzed with Microarray Suite version 5.0 (MAS 5.0) using Affymetrix default analysis settings and global scaling as normalization method. The trimmed mean target intensity of each array was arbitrarily set to 500. The reported data values represented log2 of MASS-calculated signal intensity.

In the current work, we utilized gene expression values for the expression values for the ‘_at’ probes and probes that only overlapped a single annotated transcript. Using this filtering strategy, we had gene expression levels for 19,583 genes. In order to validate our gene signature, we used published DLBCL datasets that had paired gene expression and survival outcome data available in GEO: GSE34171, GSE32918/69051 and DLBC from The Cancer Genome Atlas (TCGA; portal.gdc.cancer.gov/). Uses and the gene expression platforms for different dataset are presented in Table S3.

Identification of Genes Associated with Overall Survival

Individuals were assigned two distinct groups based on the median gene expression value from the GSE10846 dataset. Using the R package survival version 3.1-8. Kaplan-Meier curves were plotted for each group using the ‘survfit’ function and the P-values for log-rank test were calculated using the ‘survdiff’ function. P-values for all the 19,583 genes were recoded and 61 of those genes were found to be significant at P-value <=0.001, which was our threshold for this analysis.

### Development of the Prognostic Gene Signature

We developed an analysis pipeline to identify a prognostic gene signature and validate it in other DLBCL datasets. LASSO (Least Absolute Shrinkage and Selection Operator) analysis was carried out to identify a set of marker genes that could predict the overall survival using the R package glmmet version 3.0-2. For LASSO analysis only the significant genes p<0.001 (total 61 as described in the previous section) were used. 33 significant markers were identified, and relative regression coefficients were recorded for them (Table 1).

### Code Used for LASSO Regression:

set. seed(1011)

## Run Cross Validation

CV=cv.glmnet(x=as.matrix(t_Exp_data),y=y,family=“cox” ,type.measure=“C”, alpha=1, nlambda=100, parallel=T)

We then used LASSO logistic regression analysis model and 33 maker gene signatures were selected using 10-fold cross-validation with the minimum value of log (λ) −3.3 based on the 1 standard error criteria (FIG. 6). The C-index in the y-axis shows the goodness of fit in the model. The region between the vertical dashed lines represents models within one standard error of the minimum, which is the most regularized form, for the selected C-index value.

Enrichment of molecular pathways of the 33 gene signature was performed using Metascape using standard parameters (Zhou et al., 2019).

### Calculation of Risk Scores for Individuals Based on 33-Gene Signature

From Table 1, we used the coefficient value for each gene in our signature and the expression of the gene is taken from the expression matrix of the dataset. Next, we multiplied the coefficient value by its expression value and repeated this for all signature genes. Finally, we sum these individual values to get a risk score for a sample. An example is shown in Table S4. We repeated this for all individuals in the dataset.

### Validation of Prognostic Gene Signature on Additional Datasets

We used the dataset GSE10846 to identify the gene signature that is associated with OS and found significant p-value on performing survival analysis based on risk score as defined earlier on this dataset. In order to validate our gene signature, we used GSE34171, GSE32918/69051 and DLBC TCGA datasets. The risk score was calculated for all the samples as described earlier and survival analysis was done based on the median risk score value to separate the individuals into high and low risk score groups for analysis.

### Software for Statistical Analysis

For statistical analysis and graphical plotting we utilized R version 3.6.1, glmmet version 3.0-2, Survival version 3.1-8, ggsurvplot version 0.4.6, ggplot2 version 3.3.0 and ComplexHeatmap version 2.2.0. and GraphPad Prism version 8.

### RESULTS

Identification of Genes Associated with DLBCL Survival Outcomes

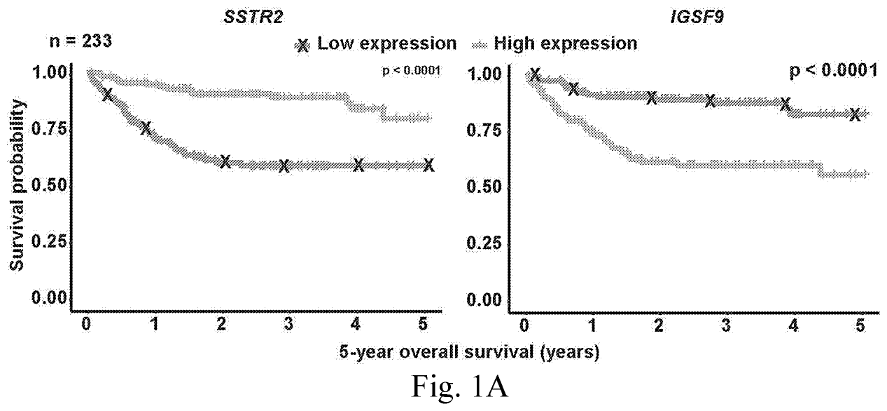

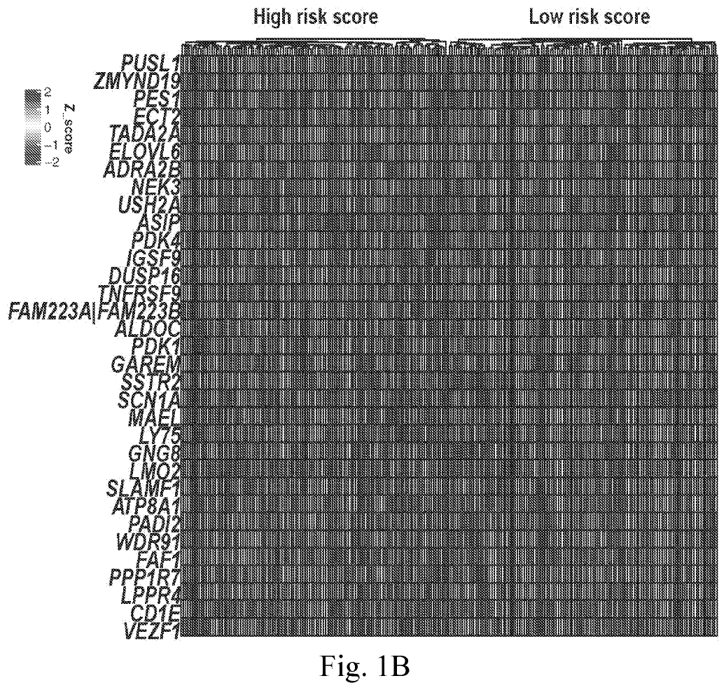

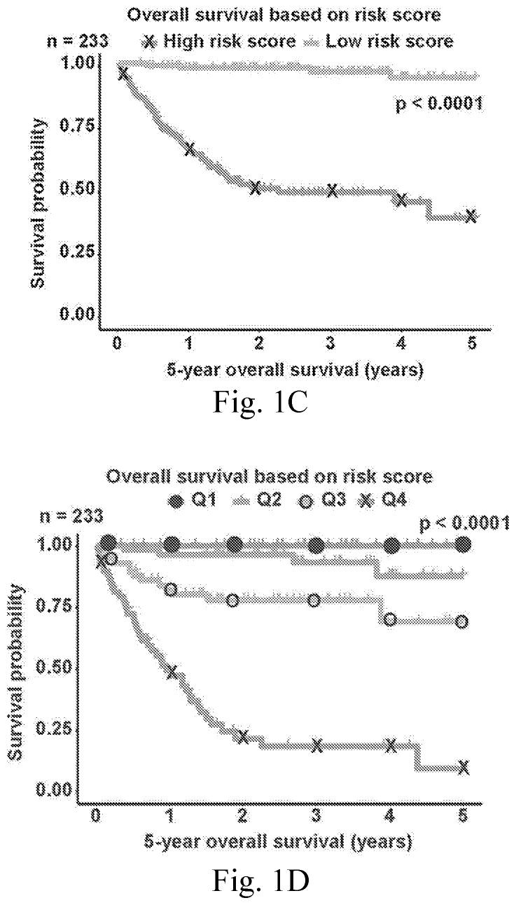

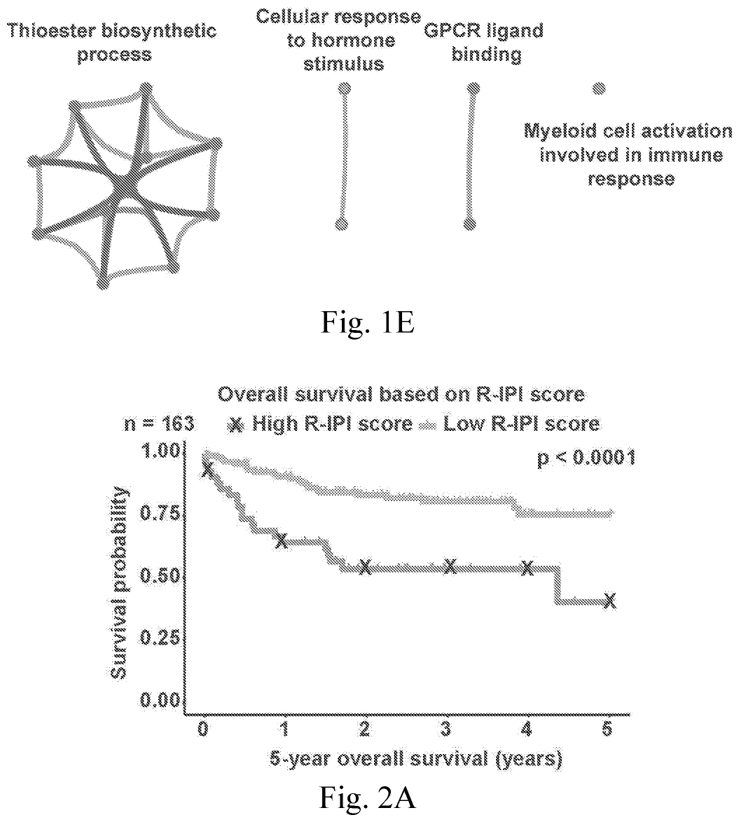

We first determined genes that were associated with overall survival in DLBCL individuals from the Lymphoma/Leukemia Molecular Profiling Project (LLMPP) cohort that consisted of de novo diagnosed patients that were treated with R-CHOP (n=233) that had tumor gene expression profiling and were monitored for clinical outcome (GSE10846). This dataset consisted of adults aged 17-92 with an average age of around 60 years old with 99 (42.5%) females and 134 (57.5%) males. We identified 1,318 genes that were significantly (p<0.05) associated with 5-year overall survival using an univariant cox regression model (Table S1). The gene that encodes the somatostatin receptor (SSTR2; p<0.0001) and the gene that encodes the immunoglobulin superfamily member 9 (IGSF9; p<0.0001) had the lowest p-values, which when individuals were separated into high or low median gene expression groups, had high or low gene expression associated with overall survival, respectively (FIG. 1A).

There were 61 genes individually associated with overall survival that had a p value <.001 using the univariant cox regression model (Table S1). We then used these 61 genes in a Lasso Multivariate Cox analysis to identify a minimal set of genes that could predict overall survival and identified a minimal set of 33 genes (Table 1). The expression levels of these 33 genes multiplied by dataset coefficients were used to develop a survival risk score for each individual (Table 1). A higher risk score equates to a higher mortality risk for individuals with DLBCL. We stratified individuals in the DLBCL cohort into high and low risk score based on the median risk score among the entire cohort and found differences in expression levels of the 33 genes between the high and low risk score groups (FIG. 1B). Next, we found that the overall survival of the high-risk group was significantly reduced compared to the low risk group (HR=0.046 (0.017-0.13 95% CI); p<0.0001; FIG. 1C). Moreover, when we stratified individuals by risk score into quartiles, the individuals in the lowest quartile of risk score (Q1) had a 100% probability of survival whereas individuals in the highest quartile (Q4) had a 9.2% OS by year five (FIG. 1D).

Using Metascape, we identified the top biological pathways and processes that were significantly over-represented in our 33 gene set: Thioester biosynthetic process (p=4.7E-5), Cellular response to hormone stimulus (p=0.002), GPCR ligand binding (p=0.003) and Myeloid cell activation involved in immune response (p=0.006) (FIG. 1E). A network plot of interacting genes showed the pathway of thioester biosynthetic process contained the most interacting nodes (9) followed by cellular response to hormones and GPCR ligand binding with the only 2 interacting nodes. Myeloid cell activation involved in immune response only had single nodes without interaction (FIG. 1E). Thus, we have identified a set of 33 genes that when their gene expression levels are assembled into a risk score can significantly predict individuals with higher and lower rates of 5-year OS.

### Gene Signature can Better Predict Survival Than R-IPI Alone

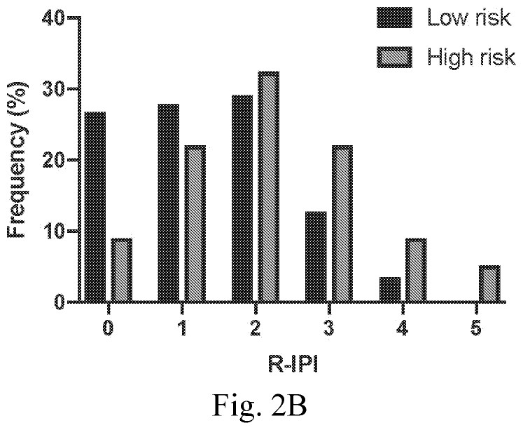

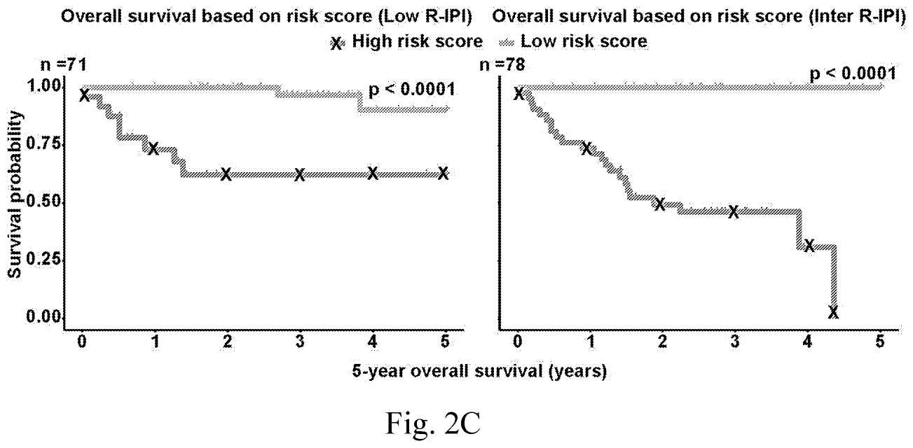

The revised International Prognostic Index (R-IPI) was developed to predict the outcome of individuals receiving rituximab with chemotherapy and subdivides individuals into 3 groups (very good, good, poor) that can predict survival. We were able to calculate the R-IPI for 163 of the 233 individuals in our dataset. As expected, individuals with low R-IPI scores had significantly improved overall survival compared to individuals with a high R-IPI score (HR=0.32 (0.17-0.58 95% CI); p<0.0001; FIG. 2A). Although using IPI alone can significantly group individuals into high and low risk, it does not group them as well as using the risk scores developed from our identified prognostic gene signature (R-IPI HR=0.32 vs risk score HR=0.046). Next, we determined the distribution of R-IPI scores of individuals with high and low risk scores derived from our prognostic gene signature (FIG. 2B). Individuals with a low risk score based on gene signature had significantly lower R-IPI scores (mean 1.38; p<.001, Wilcoxon-Mann-Whitney) compared to individuals with high risk scores (mean 2.16; FIG. 2B). However, there were individuals that had low R-IPI scores that were identified as high risk by our gene signature (9.1% of individuals with high risk score had an R-IPI of 0), and conversely, individuals that had high R-IPI scores identified as low risk by our gene signature (FIG. 2B). Next, we determined if risk scores from the prognostic gene signature could improve prediction of overall survival even in individuals with low R-IPI scores that would be expected to have superior survival as a group. We found that individuals with a high-risk score derived from the gene signature had significantly lower overall survival than individuals with low risk scores, despite having low (0-1) or intermediate (2-3) R-IPI scores (FIG. 2C). This analysis demonstrated that the risk score generated from the prognostic gene signature can better predict individuals with higher and lower overall survival even if they have favorable R-IPI scores.

Finally, we used multivariate Cox regression analysis to determine if the risk score determined by our identified gene signature could significantly predict overall survival when R-IPI or tumor molecular subtype clinical parameters were utilized as covariates. There were gene expression, tumor molecular subtype (germinal center B-cell-like or activated B-cell-like) and R-IPI scores available for 140 of the samples that we utilized for multivariate Cox regression. When molecular subtype or R-IPI were used individually as covariates or together as covariates, individuals with a low-risk score based on our gene expression signature had a significantly lower risk of death using this multivariate analysis (Table 2).

TABLE 2
Multivariate Cox regression analysis
of gene signature with covariates.

| Low risk | | | Standard | | P value |
|---|---|---|---|---|---|
| score + | Coefficient | Hazard | error | Wald | (Wald |
| covariate | beta | ratio | of HR | statistic | test) |
| Tumor subtype | −2.64 | 0.072 | 0.615 | −4.29 | 1.82E−05 |
| (ABC/GCB) | | | | | |
| R-IPI score | −2.74 | 0.065 | 0.608 | −4.51 | 6.59E−06 |
| Subtype and R- | −2.51 | 0.082 | 0.620 | −4.05 | 5.23E−05 |
| IPI | | | | | |

These data demonstrated that risk score can better predict overall survival even when using clinical parameters such as tumor molecular subtype and R-IPI score as covariates in this dataset.

### Refined Prognostic Gene Signature Based on DLBCL Molecular Subtype

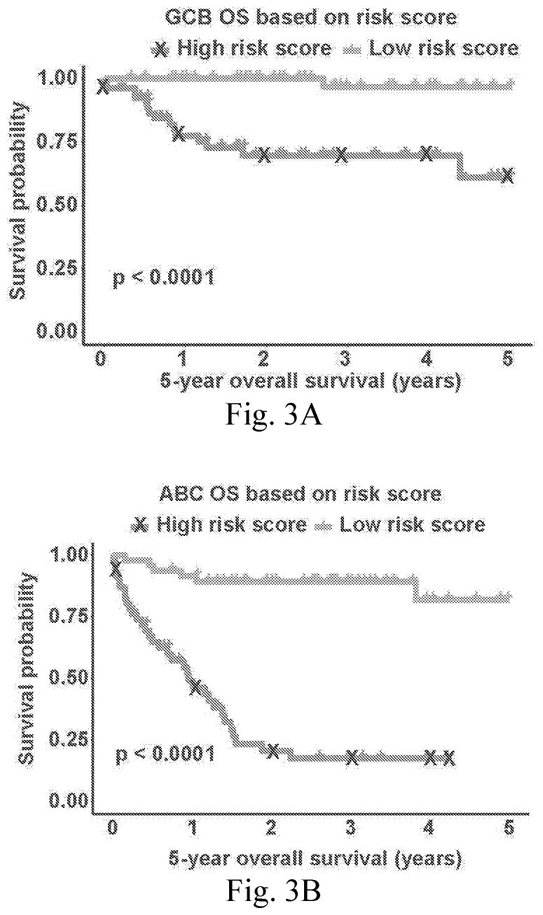

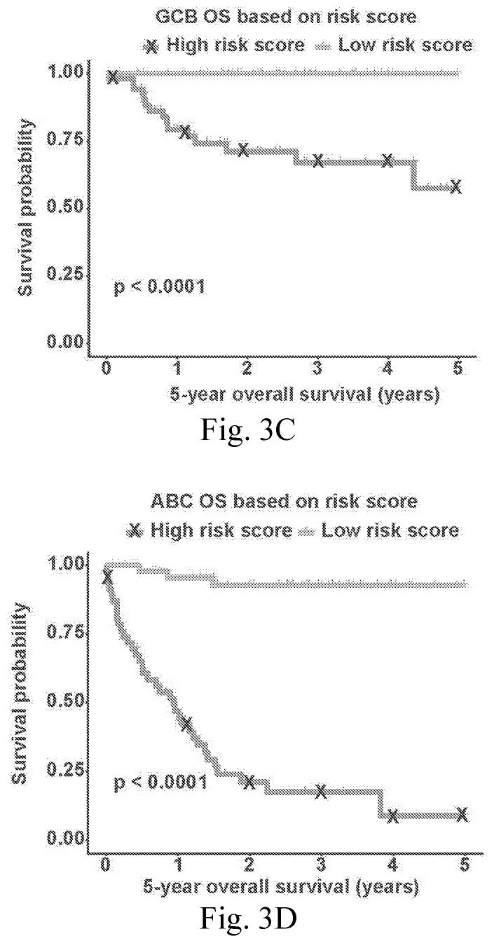

DLBCL presents as a clinically heterogenous disease, but molecular studies have identified at least two prominent molecular subclasses; GCB subclass and ABC subclass that each differ in presentation, response to therapy, and clinical outcome. We subdivided the DLBCL individuals treated with R-CHOP from the LLMPP into GCB (n=106) and ABC (n=93) subclasses and used the risk score generated from the 33 prognostic genes from the entire dataset and determined the effect of high or low risk scores on overall survival in each subclass. There were significant differences in overall survival between individuals with high or low risks scores in both GCB (HR=0.05 (0.066-0.38 95% CI); p <0.0001) and ABC (HR=0.091 (0.038-0.22 95% CI); p <0.0001) subtypes of DLBCL (FIG. 3A & 3B).

We also extracted genes associated with overall survival and used the Lasso multivariate Cox analysis to identify independent gene sets that predict overall survival for each DLBCL subtype individually. We identified an additional 12 and 16 gene panel that was significantly associated with overall survival for GCB and ABC DLBCL subtypes, respectively (Table S2). When both of these gene sets were transformed into risk scores, individuals were stratified by high and low risk score; the individuals with a low risk score had significantly higher rates of overall survival in both GCB (HR=1.1E9 (0-Inf 95% CI)) and ABC (HR=0.042 (0.013-0.14 95% CI)) of DLBCL (FIG. 3C & 3D). Similar rates of overall survival were observed using the risk scores derived from the 33 gene signature from the entire dataset or subclass-specific signatures (FIG. 3). Interestingly, there was little overlap in the gene sets that were associated with overall survival generated using all the DLBCL samples and when the two subclasses were considered independently with only 4 genes overlapping all DLBCL and ABC subclass (IGSF9, ECT2, FAFJ, USH2A), 1 gene overlapping all DLBCL and GCB subclass (ELOVL6) and no genes overlapping all GCB and ABC subclasses or all 3 gene sets. This analysis identified specific gene sets that could be applied to predict overall survival when the DLBCL subclass is known and may be more relevant for predicting survival in ABC subclasses of DLBCL.

### Evaluation of Previously Identified Prognostic Genes in DLBCL

Only one gene in our newly identified gene signature, LMO2, overlapped with three previously published DLBCL prognostic gene signatures consisting of 6, 7, or 14 gene sets (Table 3).

TABLE 3
Multivariate analysis of genes in previously
identified prognostic genes for DLBCL.

| | | | | Hazard | |
|---|---|---|---|---|---|
| | Log-rank P | Hazard | Coefficient | ratio | Lasso |
| Gene name | value | ratio | beta | pvalue | coefficient |
| 14-gene set¹ | | | | | |
| BCL6 | 0.00031974 | 0.38 | −0.98 | 0.00054 | 0 |
| CCND2 | 0.02668216 | 1.8 | 0.58 | 0.029 | 0 |
| ENTPD1 | 0.13109946 | 1.5 | 0.39 | 0.13 | 0 |
| FUT8 | 0.12303058 | 1.5 | 0.4 | 0.13 | 0 |
| IGHM | 0.97761542 | 0.99 | −0.0071 | 0.98 | 0 |
| IL16 | 0.23297869 | 0.73 | −0.31 | 0.23 | 0 |
| IRF4 | 0.08354497 | 1.6 | 0.45 | 0.086 | 0 |
| ITPKB | 0.00094581 | 0.41 | −0.89 | 0.0014 | 0 |
| LMO2 | 5.41E−05 | 0.33 | −1.1 | 0.00012 | −0.0472768 |
| LRMP | 0.01122516 | 0.51 | −0.67 | 0.013 | 0 |
| MME | 0.00077687 | 0.41 | −0.9 | 0.0012 | 0 |
| MYBL1 | 0.00293419 | 0.45 | −0.79 | 0.0038 | 0 |
| PIM1 | 0.33833034 | 1.3 | 0.25 | 0.34 | 0 |
| PTPN1 | 0.72344803 | 1.1 | 0.092 | 0.72 | 0 |
| 6 gene set² | | | | | |
| BCL2 | 0.03880542 | 1.7 | 0.54 | 0.041 | 0 |
| BCL6 | 0.00031974 | 0.38 | −0.98 | 0.00054 | 0 |
| CCND2 | 0.02668216 | 1.8 | 0.58 | 0.029 | 0 |
| FN1 | 0.64798338 | 0.89 | −0.12 | 0.65 | 0 |
| LMO2 | 5.41E−05 | 0.33 | −1.1 | 0.00012 | −0.0247045 |
| SCYA3 (CCL3) | 0.21720933 | 1.4 | 0.32 | 0.22 | 0 |
| 14-gene set³ | | | | | |
| GPNMB_1554018_at | 0.11130362 | 0.66 | −0.41 | 0.11 | 0 |
| ITPKB_1554306_at | 0.00314772 | 0.46 | −0.78 | 0.004 | −0.0139079 |
| GPNMB_201141_at | 0.21553766 | 0.73 | −0.32 | 0.22 | 0 |
| CALD1_201615_x_at | 0.27605649 | 0.75 | −0.28 | 0.28 | 0 |
| CALD1_201616_s_at | 0.11429087 | 0.66 | −0.41 | 0.12 | 0 |
| CALD1_201617_x_at | 0.08866814 | 0.64 | −0.44 | 0.092 | 0 |
| RTN1_203485_at | 0.09453336 | 0.65 | −0.43 | 0.097 | 0 |
| APOC1_204416_x_at | 0.67070185 | 0.9 | −0.11 | 0.67 | 0 |
| PLAU_205479_s_at | 0.04024081 | 0.59 | −0.53 | 0.043 | 0 |
| RTN1_210222_s_at | 0.01236298 | 0.52 | −0.66 | 0.014 | 0 |
| CD84_211192_s_at | 0.30230008 | 1.3 | 0.27 | 0.3 | 0 |
| CALD1_212077_at | 0.46502841 | 0.83 | −0.19 | 0.47 | 0 |
| CALD1_214880_x_at | 0.07202435 | 0.63 | −0.47 | 0.075 | 0 |
| ITPKB_235213_at | 0.00056591 | 0.39 | −0.94 | 0.00089 | −0.0708992 |

¹Wright et al., A gene expression-based method to diagnose clinically distinct subgroups of diffuse large B cell lymphoma. Proc Natl Acad Sci U S A 20 03; 10 0: 9991-6.
²Lossos et al., Prediction of survival in diffuse large-B-cell lymphoma based on the expression of six genes. N Engl J Med 2004; 350: 1828-37.
³Zamani-Ahmadmahmudi & Nassiri, Development of a Reproducible Prognostic Gene Signature to Predict the Clinical Outcome in Patients with Diffuse Large B-Cell Lymphoma. Sci Rep 2019; 9: 12198.

We used the previously published gene signatures to perform Lasso multivariate analysis using R-CHOP treated individuals in the LLMP dataset to evaluate their ability to predict overall survival. To calculate risk scores in our signature analysis, we multiplied the Lasso coefficient by individual genes' expression and the sum of these values for the entire gene list forms a risk score to stratify DLBCL individuals for survival analysis. In our prognostic gene list, all 33 genes were significantly associated with overall survival independently, and nonzero Lasso coefficients were used to calculate risk scores that resulted in improved prediction of overall survival (Table 1). In contrast, in all of the three previously identified gene signatures, only a single gene yielded a nonzero coefficient in each gene list, meaning risk scores could only be calculated using a single gene and thus not robust enough for further analysis using multivariate methods on this DLBCL dataset (Table 3). In the two of the gene signatures, the LMO2 gene yielded a nonzero coefficient and for the third gene set, two probes that mapped to the ITPKB gene had a nonzero coefficient. Despite not being able to calculate multivariate risk scores with these datasets, one set had 7 of 14 genes, another had 4 of 6 genes and the third had 3 of 7 genes that had significant impact on overall survival when hazard ratios were calculated individually (Table 3). Thus, while a fraction of the genes in the previously identified prognostic gene signatures were individually associated with overall survival outcomes, multivariate risk scores could not be calculated with these gene lists. Our newly identified prognostic gene signature allows superior assessment of risk of high or low overall survival when analyzing R-CHOP treated DLBCL in the LLMP dataset.

### External Validation of the Prognostic Gene Expression Risk Score

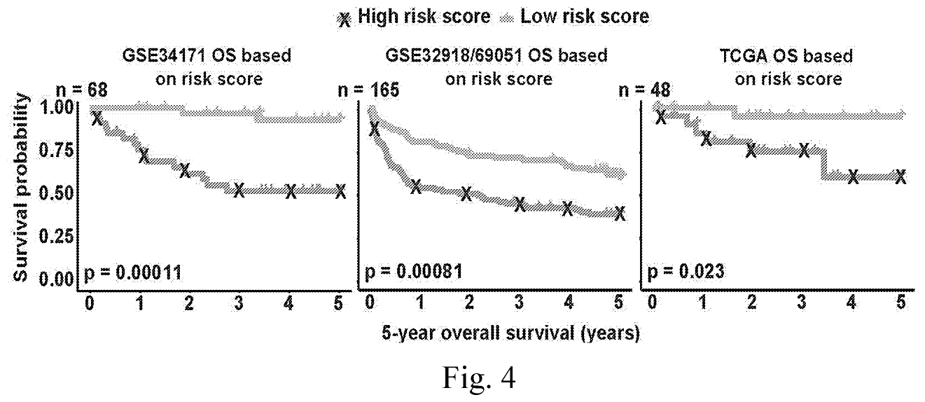

We next sought to validate our 33-gene prognostic signature in other DLBCL cohorts that had molecular profiling and clinical outcomes. Two additional studies performed microarray gene sequencing (GSE34171 and GSE32918/69051) of 68 and 165 DLBCL individuals respectively and 48 individuals with DLBCL in the Cancer Genome Atlas (TCGA) that underwent molecular profiling with next-generation sequencing (Table S3). Risk scores were calculated for each dataset using the expression of the 33 genes we identified using the LLMPP samples and individuals were stratified into high and low risk groups using the mean score as the break point. In GSE34171 (HR=0.095 (0.022-0.42 95% CI); p=0.00011), GSE32918/69051 (HR=0.5 (0.32-0.78 95% CI); p=0.00081) and TCGA (HR=0.12 (0.015-1 95% CI); p=0.023) five-year overall survival was significantly improved in individuals with a low-risk score using our gene set compared to the high-risk score individuals (FIG. 4).

### SUPPLEMENTAL TABLES

TABLE S1

| Gene name | p value | Gene name | p value |
|---|---|---|---|
| SSTR2 | 2.65E−06 | LOC642426 | 0.02224792 |
| ALDOC | 6.26E−06 | ANXA5 | 0.02229275 |
| IGSF9 | 9.19E−06 | LINC00467 | 0.02232814 |
| ATP8A1 | 2.06E−05 | RP11-805I24.3 | 0.0223508 |
| ABHD12 | 7.45E−05 | MRC1 | 0.02238107 |
| SERTAD4 | 7.68E−05 | IFNL1 | 0.02239504 |
| LY75 | 9.00E−05 | ENTPD1-AS1 | 0.0224068 |
| TADA2A | 0.000107164 | RAMP1 | 0.02241929 |
| USH2A | 0.000128985 | TCP11L2 | 0.02243167 |
| PDK4 | 0.0001327 | TTC4 | 0.02245417 |
| PUSL1 | 0.000132954 | C12orf55 | 0.02246196 |
| MAEL | 0.000144786 | C7 | 0.02248732 |
| SAPCD2 | 0.000169729 | HSD17B11 | 0.02250364 |
| TTC9 | 0.000170749 | LRRC37A5P | 0.02256458 |
| FAM223A\|FAM223B | 0.000171971 | PROSER3 | 0.02260493 |
| SNHG16\|SNORD1A\| | 0.000184692 | VEZT | 0.02261251 |
| SNORD1C | | | |
| CD1E | 0.000200923 | CEACAM19 | 0.02268652 |
| VEZF1 | 0.000213634 | CYLC2 | 0.02270968 |
| DLEU2\|MIR15A | 0.000223744 | FANCF | 0.02272932 |
| KLHL5 | 0.000229849 | MROH2A | 0.02275287 |
| LMO2 | 0.000231923 | LINC01126 | 0.0227715 |
| AK056982 | 0.000233154 | AGFG1 | 0.02282371 |
| PMM2 | 0.000273062 | LPPR5 | 0.02290759 |
| PADI2 | 0.000285203 | PLK2 | 0.02294986 |
| NIPA2 | 0.000327406 | MNX1 | 0.02295321 |
| NAB2 | 0.000344503 | CTD-2555O16.4\|MTHFD1 | 0.02296266 |
| WDR91 | 0.000352996 | GPR123 | 0.02296283 |
| LOC101928409 | 0.000361696 | MARS2 | 0.02302542 |
| JADE3 | 0.000366548 | HDAC4 | 0.02302813 |
| HENMT1 | 0.000408069 | A2MP1 | 0.02317771 |
| GNG8 | 0.000422099 | FAM83E | 0.02326478 |
| FAF1 | 0.00044069 | LOC100288893 | 0.02334731 |
| TRIM52 | 0.000461981 | ERAP1 | 0.02334736 |
| SLAMF1 | 0.000496635 | LKAAEAR1 | 0.0234283 |
| AZIN2 | 0.00051182 | TXK | 0.02350375 |
| RNF19B | 0.000512124 | CDC34 | 0.02357474 |
| RARRES2 | 0.000516069 | MX2 | 0.02368031 |
| EEPD1 | 0.000520358 | LOC100996542 | 0.02369583 |
| C3 | 0.000522632 | OR2J3 | 0.02371762 |
| ADRA2B | 0.000532255 | SLC18B1 | 0.02371874 |
| DUSP16 | 0.00053301 | UCMA | 0.02373895 |
| ASIP | 0.000556487 | ARHGAP25 | 0.02375205 |
| SCN1A | 0.000595384 | NGLY1 | 0.0237666 |
| PPP1R7 | 0.000600294 | ETF1 | 0.02384839 |
| ECT2 | 0.000626993 | LINC00487 | 0.02385189 |
| IL22RA2 | 0.000639952 | FADS3 | 0.02389167 |
| NEK3 | 0.000653004 | KIAA1586 | 0.0239119 |
| SPINK2 | 0.000691883 | EMILIN2 | 0.023929 |
| FLCN\|PLD6 | 0.000769751 | GPR150 | 0.02401061 |
| ZNF271 | 0.00079056 | PBX4 | 0.0240172 |
| SSBP3 | 0.000796249 | RP3-337H4.8 | 0.02409142 |
| PES1 | 0.000807743 | RP11-138I18.2 | 0.02410516 |
| ELOVL6 | 0.00083533 | RGPD4-AS1 | 0.02410803 |
| LPPR4 | 0.000857769 | POMP | 0.02428552 |
| CSTA | 0.000882918 | LOC102725345 | 0.02435118 |
| WFIKKN1 | 0.000890317 | ZNF565 | 0.02435814 |
| ZMYND19 | 0.000892789 | BIRC5\|EPR-1 | 0.02437635 |
| GAREM | 0.000919432 | CACNA1G | 0.0244283 |
| TNFRSF9 | 0.000942425 | ZBTB32 | 0.02445549 |
| ITPKB | 0.000945813 | CIR1 | 0.02451997 |
| PDK1 | 0.000947055 | C1QB | 0.02453533 |
| KIF26B | 0.001023272 | METTL8 | 0.02454048 |
| SLC7A11 | 0.001044817 | ZNF133 | 0.02456983 |
| CNGB3 | 0.001051551 | ETFA | 0.02477218 |
| TFCP2 | 0.001063494 | LINC00654\|LOC643406 | 0.0247789 |
| PRKCZ | 0.001077222 | ASPH | 0.02478614 |
| ARSI | 0.001098794 | SLC38A5 | 0.02495012 |
| YME1L1 | 0.001106527 | ADIPOQ | 0.02495305 |
| PTRH2 | 0.001111292 | DYNLL1 | 0.02495635 |
| FNDC1 | 0.001111988 | NTHL1 | 0.02495656 |
| NFXL1 | 0.0011123 | ARHGEF3 | 0.02497641 |
| BC045805 | 0.00121317 | PIP4K2A | 0.02499149 |
| RELB | 0.001220648 | ALG8 | 0.02500057 |
| CENPC | 0.00127394 | SERTAD4-AS1 | 0.02500388 |
| MRPL2 | 0.00137891 | MSH4 | 0.02505098 |
| LINC00954 | 0.001411903 | NME8 | 0.0250687 |
| CAPG | 0.001420873 | LINC00643 | 0.02510101 |
| METTL7B | 0.001432459 | IGLC1 | 0.02522935 |
| RXRG | 0.001436252 | TCRA\|TCRAV5.1a | 0.02526378 |
| TMEM119 | 0.001440231 | XRCC4 | 0.02530506 |
| HRSP12 | 0.001472414 | CD9 | 0.02537793 |
| CNPY3 | 0.001491909 | MMP20 | 0.02538866 |
| TANGO6 | 0.001492849 | RP3-388M5.9 | 0.02539756 |
| LOC101928283 | 0.001595741 | BATF | 0.0254769 |
| DHRS1 | 0.001615082 | GPR82 | 0.02548016 |
| EMR3 | 0.001668864 | LINC01209 | 0.02551937 |
| MTL5 | 0.001682422 | RP11-109M19.1 | 0.02558672 |
| GATA2 | 0.00169427 | CCDC144A | 0.02560606 |
| CCL8 | 0.001727951 | PAK6 | 0.0256881 |
| TMEM37 | 0.001733644 | ADAM12 | 0.02572513 |
| POLDIP3 | 0.001754113 | SLC39A13 | 0.02577066 |
| SLC1A5 | 0.001779503 | RCAN2 | 0.02577619 |
| MTUS2-AS1 | 0.001818472 | SH3YL1 | 0.02579278 |
| RGS17 | 0.001851791 | AURKAPS1\|RAB3GAP2 | 0.02582614 |
| ADAT2 | 0.001865471 | NOL3 | 0.02584195 |
| SNTA1 | 0.001965056 | CUL4A | 0.02590442 |
| BCL6 | 0.001995927 | CPSF2 | 0.02591271 |
| AC091633.3 | 0.002052761 | KIR3DX1 | 0.02595488 |
| LOC285500 | 0.002064651 | FGF11 | 0.02617466 |
| CCL27 | 0.002065541 | ENKUR | 0.02619895 |
| PP7080 | 0.002087081 | APOL5 | 0.02620997 |
| C1orf109 | 0.002101159 | DENND3 | 0.02622179 |
| MAGED2 | 0.002218188 | ZNF317 | 0.02626346 |
| FAM155A | 0.002230887 | RP11-250B2.6 | 0.02628189 |
| ZNF284 | 0.002244411 | FBXO21 | 0.02632133 |
| UBL7 | 0.002244704 | SLC22A3 | 0.02634878 |
| FBLL1 | 0.002249837 | LARGE | 0.02647975 |
| OPALIN | 0.002256065 | GFPT2 | 0.02650345 |
| SMIM15 | 0.002267178 | FOXF2 | 0.0265058 |
| AMACR/C1QTNF3- | 0.002283716 | LDHD | 0.026541 |
| AMACR | | | |
| WDR60 | 0.002345022 | PADI1 | 0.02660545 |
| RP11-53915.1 | 0.002378813 | SET\|SETSIP | 0.02667187 |
| CYP27B1 | 0.00238212 | LINC00838 | 0.02685639 |
| TBC1D7 | 0.002435714 | CDC27 | 0.02685725 |
| XK | 0.002445195 | LOC100505915 | 0.0268659 |
| LOC439951 | 0.00246376 | EBPL | 0.02691527 |
| NFRKB | 0.002528688 | ACVR2A | 0.02693815 |
| CPNE5 | 0.002615549 | ZNF608 | 0.02698643 |
| 2-Mar | 0.002621466 | SLC1A7 | 0.02701096 |
| GDPD5 | 0.00266 | ATP6 | 0.0270253 |
| RP11-245P10.8 | 0.002713779 | CTD-2292M16.8 | 0.02707403 |
| FAM50B | 0.002726937 | BEND6 | 0.02713355 |
| LOC101927278 | 0.00274053 | EGLN1 | 0.02714897 |
| MRPL3 | 0.002746459 | FAM101B | 0.02721786 |
| ESRG\|MIR4454 | 0.002756777 | LOC101060181\|ZNF44 | 0.02725109 |
| C5orf30 | 0.002798348 | TTC13 | 0.02725233 |
| RP5-1027O15.1 | 0.002853488 | GTPBP6 | 0.02733472 |
| MRPS9 | 0.002873276 | LPP | 0.02740012 |
| MYBL1 | 0.002934193 | KAT2A | 0.02741668 |
| NME1 | 0.002945188 | PLAG1 | 0.02748207 |
| RAP2A | 0.002948045 | ACTN1 | 0.02757312 |
| L1CAM | 0.002957364 | SNORD89 | 0.02760139 |
| CHCHD4 | 0.00298275 | LINC00929 | 0.0276983 |
| ING2 | 0.00305214 | ARHGAP29 | 0.02771893 |
| SLC5A5 | 0.003110177 | LARS2 | 0.02772215 |
| PNMAL1 | 0.003125233 | SLC2A13 | 0.02772598 |
| PHEX-AS1 | 0.003132924 | CHST1 | 0.02776911 |
| KCNA5 | 0.003132994 | POLR2D | 0.02778043 |
| ELL2 | 0.003217228 | RP11-452L6.1 | 0.02779897 |
| C12orf77 | 0.003260472 | ZMPSTE24 | 0.02790029 |
| SERPINF1 | 0.003261506 | RTN2 | 0.02791229 |
| KIAA1244 | 0.003289386 | FITM2 | 0.02792983 |
| TPTE2P5 | 0.003339207 | POLR1B | 0.02798706 |
| LEP | 0.003375538 | TCTN3 | 0.02799462 |
| S1PR2 | 0.003388442 | PARPBP | 0.02803087 |
| SLC12A3 | 0.003426415 | PRAME | 0.02803391 |
| C5orf51 | 0.003470166 | LOC101928927\|SNHG15\| | 0.02805852 |
| | | SNORA9 | |
| RAB7B | 0.003493788 | ITGB3 | 0.02811904 |
| SLAIN1 | 0.003534274 | OR8G1 | 0.02815551 |
| SMAD5 | 0.003537103 | CRYBA4 | 0.02816151 |
| DANCR | 0.003544965 | NUDT9P1 | 0.0281872 |
| TAAR9 | 0.003582974 | IGHA1\|IGHG1\|IGHM\| | 0.02820919 |
| | | IGHV3-23\|IGHV4-31 | |
| UGT3A1 | 0.003627377 | MBTPS1 | 0.0282307 |
| CD3EAP | 0.003659649 | BMPR1A | 0.02829506 |
| NR3C1 | 0.00366686 | LOC100507054 | 0.02833512 |
| RPS15A | 0.003731272 | HDAC2 | 0.02833606 |
| PTK2 | 0.003731412 | AHDC1 | 0.02839077 |
| CTXN3 | 0.003744738 | IDH1-AS1 | 0.02840888 |
| SLC12A8 | 0.003761647 | GALE | 0.02851181 |
| ZNF185 | 0.00376448 | GPC5 | 0.02853491 |
| LOC729680 | 0.003821901 | CRYAA | 0.02855246 |
| SLC23A2 | 0.003856869 | ZNF30 | 0.02857439 |
| ATP4B | 0.003935376 | BBS10 | 0.0286089 |
| INHBA-AS1 | 0.003964301 | FANCG | 0.02863608 |
| SCD5 | 0.004008529 | YDJC | 0.02868837 |
| QPRT | 0.004016737 | SYNDIG1 | 0.02874439 |
| MASIL | 0.004030324 | CEP55 | 0.02880487 |
| ENDOD1 | 0.004038981 | ODC1 | 0.02881097 |
| NAT9 | 0.004077202 | DKKL1P1\|DKKL1P1 | 0.02883769 |
| TTC27 | 0.004109962 | CTC-523E23.1 | 0.02883774 |
| GRPEL1 | 0.004154904 | C10orf95 | 0.0288484 |
| USP20 | 0.004174867 | LOC100127974 | 0.02898311 |
| CCL18 | 0.004189416 | BEAN1 | 0.02899768 |
| ZBED6CL | 0.00429736 | NAGS | 0.0290783 |
| TMEM97 | 0.004316206 | RP11-108B14.5 | 0.02913054 |
| SCN2A | 0.004358074 | RGS13 | 0.0291586 |
| HPDL | 0.004397106 | BUB3 | 0.02917303 |
| ZFP37 | 0.004449551 | CEP72 | 0.02917356 |
| SLA | 0.00447649 | LOC101927990 | 0.02934247 |
| SSBP2 | 0.004515583 | CCT6B | 0.02935108 |
| NYAP2 | 0.004537742 | ZNF200 | 0.02938241 |
| ME2 | 0.004542515 | CYB561A3 | 0.02944464 |
| FKBP11 | 0.004553044 | LOXL1 | 0.02959285 |
| PTGIR | 0.00456582 | ATP13A3 | 0.02960762 |
| TRAF1 | 0.004587534 | HSDL1 | 0.02961564 |
| PCDH9 | 0.004587629 | TCAP | 0.02967586 |
| EIF2A | 0.00468065 | RP1-58B11.1 | 0.02968041 |
| MIR6872\|SEMA3B | 0.004697484 | VSTM1 | 0.02968541 |
| PRPSAP2 | 0.004760217 | APLF | 0.02971922 |
| FYTTD1 | 0.004768343 | RPTOR | 0.02973103 |
| TRIB1 | 0.004775666 | LPCAT4 | 0.0297554 |
| TMCC1-AS1 | 0.004818521 | ADD3 | 0.02975905 |
| UBE2V2P3\|UBE2V2P3 | 0.004819176 | ULBP3 | 0.02978355 |
| SEL1L3 | 0.004839362 | RDM1 | 0.02978923 |
| OXR1 | 0.004846244 | ASL | 0.02987989 |
| NT5DC4 | 0.00485631 | RRP9 | 0.02991211 |
| FCGR3B | 0.004880709 | LRFN2 | 0.02991234 |
| ERV3-2 | 0.005033371 | SORL1 | 0.02992827 |
| SRM | 0.005193772 | NOD2 | 0.02994761 |
| KLHL8 | 0.005198324 | LOC101928255 | 0.03000014 |
| C19orf83 | 0.005201643 | ARID5A | 0.03001643 |
| MTERF4 | 0.00525902 | RP11-799D4.4 | 0.03010441 |
| SNHG4 | 0.005392169 | WIPF3 | 0.03010504 |
| MIR100HG | 0.005431643 | CCT7 | 0.03011047 |
| SCG5 | 0.005473493 | FRMD3 | 0.03020043 |
| AAMP | 0.005581542 | LOC101926916 | 0.03023572 |
| ZMYM6 | 0.005588383 | P2RY14 | 0.03039265 |
| ACKR3 | 0.005634956 | CLNK | 0.03040388 |
| OR4C1P | 0.005643368 | C5orf58 | 0.03042226 |
| PGP | 0.005681559 | LOC101928554 | 0.03042862 |
| PRKCDBP | 0.005747155 | C10orf91 | 0.03043482 |
| C3orf80 | 0.005788786 | KANSL3 | 0.03045898 |
| PANK1 | 0.005799597 | RP11-349E4.1 | 0.03048432 |
| RBP7 | 0.005810639 | CRLS1 | 0.03049233 |
| SLC35A2 | 0.005822049 | WEE1 | 0.03053531 |
| TRIM16 | 0.005846387 | TG | 0.03059376 |
| PTPLAD2 | 0.005873711 | AC005523.2 | 0.0306349 |
| DNAJB2 | 0.005916139 | RELT | 0.03068954 |
| PVALB | 0.005922225 | AMH\|MIR4321 | 0.03075629 |
| ADTRP | 0.005954345 | FAM76B | 0.03076464 |
| SLIT2 | 0.005956257 | CCDC126 | 0.03078251 |
| FOXN3 | 0.006027997 | GBAP1\|LOC100510710 | 0.03084865 |
| MED16 | 0.006044686 | SIGLEC15 | 0.03086782 |
| RABIF | 0.006144046 | JAM3 | 0.03089102 |
| CANX | 0.006148519 | ZNF341 | 0.03090795 |
| UBE3C | 0.006194359 | RPPH1 | 0.03097006 |
| SLC2A6 | 0.006213718 | BETIL | 0.03099822 |
| PSMD11 | 0.006244456 | GPR155 | 0.03100504 |
| PNPT1 | 0.006269092 | PLCL1 | 0.03101194 |
| COA7 | 0.006317704 | CTD-2520113.1 | 0.03116272 |
| RIT1 | 0.006369805 | GNPTAB | 0.03120769 |
| ALPK1 | 0.006379309 | LINC00242 | 0.03122066 |
| ANKRD13B | 0.006400972 | GALK2 | 0.03123799 |
| RGS4 | 0.006434773 | ZNF532 | 0.03135587 |
| C1orf162 | 0.006439884 | GHRL | 0.03135739 |
| TNFAIP8L1 | 0.006469341 | ST6GALNAC2 | 0.03139438 |
| STAG3 | 0.00653712 | LRP12 | 0.03146135 |
| TIMP1 | 0.006549729 | ACOT13 | 0.03148888 |
| CTH | 0.006568392 | GPRC5C | 0.03154785 |
| HSPA12A | 0.006610387 | CCDC186 | 0.03154889 |
| LSAMP | 0.006621421 | FRY | 0.03156262 |
| ICOSLG | 0.00670443 | RPP38 | 0.0315971 |
| LOC100288911 | 0.006744418 | MRPL40 | 0.03164812 |
| BC028044 | 0.006779762 | POLR3G | 0.03167352 |
| VPREB1 | 0.006781758 | MPDZ | 0.03168157 |
| MED12L | 0.006839156 | ART3 | 0.03172543 |
| mir-223 | 0.00688361 | ENO2 | 0.03175024 |
| LOC152586 | 0.006903196 | ZNRF2 | 0.03178091 |
| MIR3658\|UCK2 | 0.006909989 | TMEM163 | 0.0318236 |
| C10orf2 | 0.007005019 | PLIN4 | 0.03194293 |
| LINC00965 | 0.00700697 | PPIH | 0.03196428 |
| SPINK5 | 0.007016699 | CCT5 | 0.03196468 |
| SNX24 | 0.007097756 | TRAPPC2 | 0.03197362 |
| POU6F1 | 0.007123665 | RP11-464F9.20 | 0.03202176 |
| ELOVL2-AS1 | 0.007133464 | RP11-124L9.5 | 0.0320386 |
| AUTS2 | 0.00713726 | CCDC14 | 0.03207956 |
| NTPCR | 0.007152776 | MECR | 0.03209982 |
| SLC16A1-AS1 | 0.007205221 | RP11-498E2.7 | 0.03213596 |
| HMX2 | 0.007259255 | MRTO4 | 0.03214145 |
| CD58 | 0.007261967 | LOC101928731 | 0.0321545 |
| REL | 0.007368934 | PIGH | 0.03237721 |
| KLHL22 | 0.007380695 | RP11-164P12.3 | 0.03245967 |
| SSU72P8\|SSU72P8 | 0.007390495 | PTK2B | 0.03267553 |
| ZFAND5 | 0.00740429 | LAYN | 0.03271927 |
| EPS15 | 0.007430456 | LOC102725017 | 0.032854 |
| CTA-250D10.23 | 0.007442906 | APTR | 0.03289516 |
| SGCD | 0.007452944 | RYR1 | 0.03294952 |
| TRAPPC6B | 0.00747354 | POTEKP | 0.03295118 |
| RP13-487P22.1\|UBE3A | 0.007488325 | LBP | 0.0329695 |
| SMIM13 | 0.00749873 | AKR1B1 | 0.03299435 |
| IZUMO4 | 0.007536304 | SMG7 | 0.0330747 |
| CTB-43E15.1 | 0.007564589 | NDUFS2 | 0.03312129 |
| GRIP2 | 0.007595767 | MLYCD | 0.03312393 |
| CEBPA | 0.007605628 | RBM48 | 0.03312849 |
| MXRA5 | 0.007616897 | SEC61G | 0.03319423 |
| LOC103344931 | 0.007638251 | LINC00312 | 0.03319964 |
| TRH | 0.007658352 | SIGMAR1 | 0.03320738 |
| SLC35F2 | 0.007693407 | USB1 | 0.033217 |
| SURF2 | 0.007697377 | BTBD11 | 0.03322464 |
| LOC102724517\|NLK | 0.007834684 | KIAA1671 | 0.0332557 |
| MMP2 | 0.007834917 | LOC101060004 | 0.0333046 |
| MIB1 | 0.0079506 | ACACB | 0.03333232 |
| LOC101928211 | 0.008035608 | ATP6V1H | 0.03342454 |
| ASB13 | 0.00803799 | AP2A2 | 0.03356429 |
| ASXL3 | 0.008054017 | FAM9B | 0.03362304 |
| LOC285812 | 0.008076991 | FAM213B | 0.03365146 |
| HK2 | 0.008166461 | TRIM55 | 0.03367874 |
| AC005224.2 | 0.008181339 | PSPC1 | 0.03368788 |
| KLHL21 | 0.008230203 | CSNK1E\|CSNK1E\| | 0.03372007 |
| | | LOC400927 | |
| ZCCHC18 | 0.008262141 | RFK | 0.03372703 |
| SRD5A3 | 0.008274207 | SLC25A17 | 0.03377671 |
| SPR | 0.008328591 | PDX1 | 0.03379076 |
| LYN | 0.008349168 | DLG1-AS1 | 0.03385465 |
| RNASEH2C | 0.008394623 | BDKRB1 | 0.03389264 |
| LRRTM4 | 0.008448169 | LOC400548 | 0.03393807 |
| LGI2 | 0.008489044 | RPS6KA6 | 0.0339722 |
| CLPP | 0.008501357 | C6orf141 | 0.03398027 |
| TMEM255A | 0.00852142 | FKBP7 | 0.03402237 |
| IFRD2 | 0.008606936 | CTD-2008P7.1 | 0.03403708 |
| LA16c-83F12.6 | 0.008683233 | ZNF564 | 0.03411921 |
| C11orf80 | 0.00873786 | TBX18 | 0.03414331 |
| MALT1 | 0.008803311 | IL12A | 0.03417362 |
| LINC00599\|MIR124-1 | 0.008885486 | NT5DC1 | 0.03419361 |
| ROBO1 | 0.008932768 | HSD17B4 | 0.03430198 |
| IKBKE | 0.009057175 | SLC2A8 | 0.03435864 |
| FAM83G | 0.009085039 | ZNF706 | 0.03437216 |
| LINC00474 | 0.009127871 | PDE5A | 0.03442712 |
| CENPVP1\|CENPVP2 | 0.0091326 | LOC101928865 | 0.03447163 |
| USP30 | 0.009139747 | PRKCD | 0.03448265 |
| LECT2 | 0.009222669 | LOC100507560 | 0.03452578 |
| LOC101927380 | 0.009238236 | LOC101927131 | 0.03456707 |
| GK5 | 0.009263344 | EHBP1L1 | 0.03456934 |
| RNASE6 | 0.00926369 | CD36 | 0.03457985 |
| ZFP3 | 0.009269966 | LYPD4 | 0.03460325 |
| PTAFR | 0.009278372 | H2BFXP | 0.03461494 |
| C1orf158 | 0.00929144 | TCF21 | 0.03468013 |
| POLR2L | 0.009302317 | PAX6 | 0.03468158 |
| C19orf26 | 0.009330123 | TTLL7 | 0.03470921 |
| LOC158402\|RP11- | 0.009351268 | KCNE1L | 0.03473298 |
| 401.2 | | | |
| CDH2 | 0.009376561 | KCTD2 | 0.03490384 |
| NET1 | 0.0094175 | KLHL23\|PHOSPHO2- | 0.03493428 |
| | | KLHL23 | |
| MICAL2 | 0.009420854 | C17orf99 | 0.03493892 |
| SMARCAL1 | 0.009458059 | LOC101928943 | 0.03498237 |
| TFIP11 | 0.009464497 | CACNG1 | 0.035003 |
| AP000462.1 | 0.009513966 | BPGM | 0.03501057 |
| CLIC6 | 0.009526054 | AFG3L1P | 0.03502141 |
| RP11-52A20.2 | 0.009551284 | MAOA | 0.03508048 |
| C9orf91 | 0.009560364 | SMIM12 | 0.03509937 |
| OLFM1 | 0.00958553 | MIR21\|VMP1 | 0.03514459 |
| EXO1 | 0.009652087 | GPR32 | 0.03522441 |
| SIGLEC1 | 0.009664088 | ADRA2A | 0.03531876 |
| RIMKLA | 0.009670965 | RAB25 | 0.03532398 |
| CADM4 | 0.009691831 | AEBP2 | 0.03534121 |
| AQP11 | 0.009713547 | BCAS1 | 0.03536235 |
| SLC16A9 | 0.009713955 | TXNDC12 | 0.03536407 |
| KIRREL3-AS3 | 0.009761277 | BC042022\|LOC100506331 | 0.03540673 |
| NEDD4L | 0.009761981 | BC045559 | 0.0354705 |
| LINC00301 | 0.009848862 | FSD2 | 0.03557758 |
| MASP1 | 0.009869155 | RP11-217B1.2 | 0.03558935 |
| POLD4 | 0.009920482 | HAVCR1P1 | 0.03570961 |
| MATR3 | 0.010002942 | CYB561 | 0.03576182 |
| CCL23 | 0.010056778 | MAML3 | 0.03577064 |
| NDC80 | 0.01012741 | NPEPL1 | 0.0359141 |
| VSIG4 | 0.010137159 | CORO2B | 0.0359486 |
| DCXR | 0.01014116 | DKFZP434F142 | 0.0359596 |
| PANK2 | 0.01018978 | RP11-486G15.2 | 0.035965 |
| OTOS | 0.010191379 | BC042029 | 0.03599452 |
| AGPAT5 | 0.01025786 | IGLV1-44 | 0.0360111 |
| R3HDM4 | 0.010292805 | POR | 0.03602793 |
| CRIP2 | 0.010419841 | PRR15L | 0.03605257 |
| RCCD1 | 0.010428936 | ITPK1-AS1 | 0.03605594 |
| FABP4 | 0.010449507 | PGLYRP4 | 0.03606923 |
| AFF3 | 0.010449515 | EYA4 | 0.03607522 |
| IL22RA1 | 0.010515697 | PRMT6 | 0.03609047 |
| AGAP4 | 0.010563136 | LOC100507630 | 0.0361237 |
| CALML5 | 0.010567977 | SLC16A10 | 0.03616523 |
| GATAD2B | 0.010597399 | TTC36 | 0.03618921 |
| Clorf64 | 0.010600671 | GPIHBP1 | 0.03622877 |
| RP11-18I14.11 | 0.010637074 | TREM1 | 0.03624724 |
| PIK3C2A | 0.010647241 | CDC7 | 0.03625117 |
| BRAP | 0.01065425 | PRO2214 | 0.03628356 |
| PMEPA1 | 0.010654336 | KLHDC9 | 0.03636641 |
| DUSP7 | 0.01066058 | TMEM68 | 0.03647079 |
| FBLN1 | 0.010679971 | CIC | 0.03647096 |
| LOC101928728 | 0.010689338 | LIMS1\|LIMS3\|LIMS3L | 0.03652002 |
| PCM1 | 0.010781401 | RP11-443C10.1 | 0.03652274 |
| HORMAD2 | 0.010804133 | PSMD8 | 0.03655349 |
| LOC101928955 | 0.010826126 | GAS2L2 | 0.03655751 |
| POLE2 | 0.01085255 | PTPN14 | 0.03656616 |
| ERICH1-AS1 | 0.01085968 | IRF2BP1 | 0.03657209 |
| DQ582785 | 0.010864889 | MAST3 | 0.03661174 |
| STARD10 | 0.010879413 | ALOX5AP | 0.03667012 |
| BIRC5 | 0.011008683 | NMUR2 | 0.03669497 |
| LOC100506558\|MATN2 | 0.011029039 | NPAS2 | 0.0367203 |
| HIRA | 0.01106338 | TRIM69 | 0.03695061 |
| TNFRSF10A | 0.011070673 | FLJ11710 | 0.03695547 |
| CAND2 | 0.011121048 | ADAM30 | 0.0369855 |
| IER2 | 0.01117094 | IFITM10 | 0.03699518 |
| GPX3 | 0.01117651 | FXR1 | 0.0370559 |
| LRMP | 0.011225158 | MTFMT | 0.03720042 |
| FABP6 | 0.011241325 | ZNF593 | 0.0372317 |
| RP11-342L8.2 | 0.011246564 | INTS8 | 0.03732303 |
| FADS2 | 0.011275335 | RGS12 | 0.03734939 |
| DUSP14 | 0.011280031 | MAP9 | 0.03735868 |
| C11orf42 | 0.011405021 | SALL1 | 0.03736931 |
| DEGS1 | 0.011407579 | NDUFS5\|RPL10 | 0.03737095 |
| PRMT5 | 0.011427252 | SCARA5 | 0.0374337 |
| SLITRK6 | 0.011478037 | PIWIL1 | 0.03743898 |
| BCAP29 | 0.011528298 | SEC61A2 | 0.03747535 |
| ZCCHC7 | 0.011568668 | SMIM22 | 0.0376005 |
| CCR7 | 0.01158803 | DPF3 | 0.03763668 |
| ZNF891 | 0.011663122 | TRIM26 | 0.03775393 |
| ZNF852 | 0.011665442 | STRADB | 0.03775949 |
| RRAS2 | 0.01167682 | VSIG10 | 0.03787563 |
| TMTC3 | 0.011699622 | COL8A2 | 0.03795356 |
| LILRA1 | 0.011702861 | ATG7 | 0.03801912 |
| EREG | 0.01171933 | ZNF48 | 0.03801997 |
| BC040646\|RP11- | 0.01172122 | HIST1H1C | 0.03803816 |
| 732A21.2 | | | |
| SLC5A12 | 0.011734188 | TOMM70A | 0.03826965 |
| TRIB3 | 0.011828908 | TTTY8\|TTTY8B | 0.03840851 |
| GTF3C2-AS1 | 0.011831844 | RPP40 | 0.03849938 |
| SLC25A14 | 0.011868952 | ADORA2B | 0.03850401 |
| FAM65A | 0.011886116 | DDI1 | 0.03851648 |
| FMOD | 0.011927937 | GPR124 | 0.03863169 |
| ATP5SL | 0.011991773 | SERPINB9 | 0.03865375 |
| RASGRP4 | 0.01205019 | FMNL3 | 0.0386943 |
| ADNP | 0.012113632 | IDI2 | 0.03871259 |
| ZBTB8A | 0.012153531 | OR52D1 | 0.03872515 |
| LOC102723678\| | 0.01221248 | LINC00930 | 0.03881623 |
| LOC102723709 | | | |
| MFSD12 | 0.012235271 | TBC1D8 | 0.03891148 |
| FGD2 | 0.01227504 | WNT7A | 0.03891476 |
| ZXDB | 0.012333976 | MS4A1 | 0.03906609 |
| CAMK2A | 0.012344689 | LOC100507351 | 0.03911207 |
| DAAM1 | 0.012383894 | RP11-642D21.1 | 0.03912848 |
| KRTAP9-3 | 0.012390965 | BC017209 | 0.03913225 |
| CPT1A | 0.012393121 | LGI1 | 0.03914198 |
| RP5-1031D4.2 | 0.012505904 | PTGER2 | 0.03915345 |
| ZBTB38 | 0.012514074 | TBC1D8B | 0.0391641 |
| KCNV2 | 0.012532636 | EXOSC5 | 0.0391744 |
| SYNPR | 0.01260965 | IRF8 | 0.03923485 |
| SNORA29\|TCP1 | 0.012639104 | GLCCI1 | 0.03927365 |
| UROC1 | 0.012648057 | PRO1483 | 0.03928796 |
| ZPBP2 | 0.012732449 | GNB2L1\|SNORD95\| | 0.03938826 |
| | | SNORD96A | |
| RP11-134G8.8 | 0.012738173 | ZNF396 | 0.03939759 |
| C3orf65 | 0.012782505 | SFTA1P | 0.03944591 |
| PIP | 0.01283817 | NOX4 | 0.03945025 |
| PRR19 | 0.012865053 | ILDR1 | 0.03948158 |
| CHFR | 0.012942682 | DOCK9 | 0.03948279 |
| HOXA10 | 0.012950164 | FCGR2C | 0.03956104 |
| KCNQ1-AS1 | 0.013000373 | LINC00280 | 0.03969872 |
| SNHG3\|SNORA73A | 0.013022154 | HESX1 | 0.03969944 |
| SCO2 | 0.013070451 | SCNN1D | 0.03970601 |
| PERM1 | 0.013082439 | ADM | 0.03982167 |
| LTBP2 | 0.013189732 | NLRP4 | 0.03984103 |
| HOXC12 | 0.013266092 | GNL3\|SNORD19B | 0.03992126 |
| ACCS | 0.013333864 | HCRTR1 | 0.03994897 |
| SNHG7\|SNORA17\| | 0.013379147 | FOXN4 | 0.03997831 |
| SNORA43 | | | |
| CXCR4 | 0.013381645 | KRTAP5-9 | 0.03998282 |
| PCDHGA4 | 0.013392393 | STIM2 | 0.04004271 |
| ALPK2 | 0.013499208 | LINC00652 | 0.0400734 |
| FAM162B | 0.01350376 | SGK1 | 0.04011577 |
| EHF | 0.01351198 | AK091028\|GMDS-AS1 | 0.04012093 |
| SBNO2 | 0.01353705 | RP11-5C23.1 | 0.04015946 |
| RNASE2 | 0.013595293 | ANKRD9 | 0.04021724 |
| MRPL1 | 0.013624449 | RPS17P5\|RPS17P5 | 0.04023694 |
| HCG4B | 0.013682013 | MFI2 | 0.0402673 |
| C11orf68 | 0.013781986 | ZNF83 | 0.04027798 |
| RBFOX2 | 0.013821323 | HTR5A | 0.04035595 |
| MANBAL | 0.013901553 | RP1-265C24.8 | 0.04039774 |
| RAB27B | 0.01390921 | CSMD1 | 0.04042973 |
| CD82 | 0.014005982 | MPI | 0.04048319 |
| KLHL6 | 0.014044559 | LINC00511\|LINC00673 | 0.04049926 |
| ABHD14A- | 0.014072581 | POM121L8P | 0.04050973 |
| ACY1\|ACY1 | | | |
| ISL2 | 0.014095721 | CD1D | 0.04055555 |
| FAM9A | 0.014114101 | UAP1 | 0.04056554 |
| DECR1 | 0.014121529 | TAS2R40 | 0.04057733 |
| LOC101927263 | 0.014125095 | FCHO1 | 0.04066731 |
| LLGL1 | 0.014144438 | ELOVL5 | 0.0406908 |
| U91328.2 | 0.014210423 | DHDH | 0.04069694 |
| LOC100127955\| | 0.014291808 | NCOA1 | 0.04075703 |
| LOC100128374 | | | |
| S100A9 | 0.01434393 | ABCC4 | 0.04076544 |
| CHST7 | 0.014362926 | DCAKD | 0.04085831 |
| RRS1-AS1 | 0.014371376 | CRB3 | 0.04086265 |
| SLAMF7 | 0.014401003 | LINC00690 | 0.0408745 |
| FCRL3 | 0.014414282 | TET2 | 0.04094352 |
| BNIP3L | 0.014419161 | SLC30A8 | 0.04095819 |
| FKSG29 | 0.014465272 | VNN3 | 0.0410418 |
| VPREB3 | 0.014577465 | RBP5 | 0.04123747 |
| AC016831.7 | 0.014607963 | POC5 | 0.04147688 |
| LOC728196 | 0.014628955 | IGSF22 | 0.04148601 |
| ZSWIM5 | 0.014652847 | C1orf210 | 0.04166618 |
| FOXA2 | 0.014672878 | ZNF286A\|ZNF286B | 0.04175061 |
| MAP2K1 | 0.01469479 | LOC100505774 | 0.04180159 |
| LOC100289230 | 0.014754577 | TTBK2 | 0.04185507 |
| ZNF469 | 0.014815743 | BC037861\|CTD-2036P10.3 | 0.0418581 |
| LOC100506047 | 0.014834187 | CCDC147 | 0.04203209 |
| LINC00936 | 0.014872461 | AC010524.4 | 0.04204364 |
| BHLHB9 | 0.014877769 | LRRK1 | 0.04220556 |
| VPS36 | 0.014943549 | LQFBS-1 | 0.042212 |
| MT1M | 0.014978357 | PET112 | 0.04226354 |
| DEF8 | 0.015084266 | TXN | 0.04228826 |
| RP4-710M16.1 | 0.015084283 | LBX1 | 0.04230738 |
| IGFBP2 | 0.015094028 | LIPH | 0.04233096 |
| SPCS3 | 0.015194607 | LINC01398 | 0.04241472 |
| GIP | 0.015238086 | C1orf122 | 0.04246014 |
| ERP44 | 0.015276552 | SOGA1 | 0.04248014 |
| UBL4B | 0.015286533 | CYP2J2 | 0.04257661 |
| ABHD6 | 0.015294929 | PTGES3 | 0.04259463 |
| LSP1 | 0.015303205 | RASA3 | 0.04267283 |
| CC2D2A | 0.015412655 | LOC219688 | 0.04267934 |
| FOXP1 | 0.015418748 | MBLAC2 | 0.04269049 |
| SAMD4A | 0.015419831 | PRPF18 | 0.04271112 |
| HIST1H3C | 0.015469672 | ZNF16 | 0.04276124 |
| LAMP3 | 0.015488803 | SH3GL1P2 | 0.04277045 |
| CDK14 | 0.015490618 | CHKB-AS1 | 0.04277837 |
| COL28A1 | 0.015495866 | LOC100506870\|LOC283140 | 0.04278072 |
| FLJ31713 | 0.015515647 | LOC101927690 | 0.04280609 |
| MRPS16 | 0.015523047 | CTD-3193013.1 | 0.04283894 |
| CEP83 | 0.015594931 | OR5E1P | 0.04293977 |
| DIP2C | 0.015595064 | NFE2L3 | 0.04296634 |
| TNK2 | 0.015597441 | LOC100507459 | 0.04311212 |
| BDH2 | 0.015617111 | CTD-2076M15.1 | 0.04311931 |
| SHISA8 | 0.01563898 | LINC01431 | 0.04313824 |
| ODF3L1 | 0.01570733 | N4BP2 | 0.04314698 |
| ZNF84 | 0.015708172 | ZSCAN12 | 0.04315875 |
| C4orf46 | 0.015716864 | LOC100508046\|LOC101929572\| | 0.04315922 |
| | | POTEH-AS1 | |
| TIMM50 | 0.015768843 | C15orf32 | 0.04317753 |
| C15orf61 | 0.015843366 | LNPEP | 0.04318913 |
| COQ7 | 0.015865181 | MTFR2 | 0.04321786 |
| DPPA3\|DPPA3P2\| | 0.015865849 | GLTSCR1L | 0.04329944 |
| LOC101060236 | | | |
| ELMO2 | 0.015982053 | TDRKH | 0.04333931 |
| BMF | 0.016018485 | NDUFA3 | 0.04335608 |
| RP11-359K18.3 | 0.016054525 | BC040833 | 0.04338894 |
| IKZF4 | 0.016058833 | MED7 | 0.04343125 |
| NEUROD6 | 0.016122301 | STRIP2 | 0.04344706 |
| C4orf26 | 0.016167665 | CUL5 | 0.04354901 |
| RP11-216L13.19 | 0.016178156 | LOC102724508 | 0.04355549 |
| LRFN4 | 0.016235028 | WARS2 | 0.04368602 |
| LINC00996 | 0.016248533 | EDA2R | 0.04369356 |
| SLC2A1-AS1 | 0.01625693 | TTC21A | 0.0437715 |
| SPRY4-IT1 | 0.016435205 | TRMT5 | 0.04379342 |
| STT3B | 0.016438938 | RNGTT | 0.04381672 |
| MEF2D | 0.016477684 | C19orf44 | 0.0438555 |
| H2AFY2 | 0.01648114 | ADH1B | 0.04387809 |
| NDOR1 | 0.016526628 | GPR6 | 0.04388855 |
| NIT2 | 0.01654515 | HDGFRP2 | 0.04389353 |
| CHD1 | 0.016585505 | GRM6 | 0.04396815 |
| CAMKMT | 0.016604613 | TTTY6\|TTTY6B | 0.04403364 |
| SPIC | 0.016616683 | CTPS1 | 0.04408911 |
| KRTAP1-5 | 0.016748722 | KIAA1147 | 0.04410076 |
| USP46 | 0.016786275 | RNF5\|RNF5P1 | 0.04410436 |
| LOC100287610\|ZNF717 | 0.016822217 | ZC2HC1B | 0.04415609 |
| BFSP2 | 0.016839148 | CBX5 | 0.04418184 |
| LOC101929910\|LOC613037\| | 0.016869962 | LOC101060521\|POLR3E | 0.04418589 |
| NPIPA5\|NPIPB11\| | | | |
| NPIPB3\|NPIPB4\|NPIPB5\| | | | |
| NPIPB8 | | | |
| NME1-NME2\|NME2 | 0.016913373 | C10orf67 | 0.04424864 |
| MTMR9 | 0.016921111 | CCNG2 | 0.04433927 |
| ZNF782 | 0.016997503 | MAPK8 | 0.04440076 |
| KCNA3 | 0.017043571 | IGF2BP3 | 0.0444342 |
| LINC00933 | 0.017072427 | CHRDL2 | 0.04447774 |
| RP11-143K11.1 | 0.017116949 | RP4-794H19.1 | 0.04452828 |
| MTHFD1 | 0.017127645 | SSFA2 | 0.04458851 |
| SYNGR2 | 0.01713541 | SYN3 | 0.04459895 |
| ALMS1P | 0.017154175 | ITGB6\|LOC100505984 | 0.04461628 |
| HMGB3P30\|HMGB3P30 | 0.017162708 | PRORSD1P | 0.04462932 |
| SGMS1 | 0.017184511 | WNT9B | 0.04468841 |
| PXMP4 | 0.017186448 | LOC101928748 | 0.04478951 |
| WDR43 | 0.017210571 | OR10D3 | 0.04479729 |
| LINC00877 | 0.017227876 | PTGDR2 | 0.04482109 |
| ZFP36L2 | 0.0172487 | CEBPA-AS1 | 0.04484474 |
| TSSK3 | 0.017293992 | FAM138A\|FAM138B\|FAM138C\| | 0.04484741 |
| | | FAM138D\|FAM138E\| | |
| | | FAM138F | |
| RP11-490G2.2 | 0.017315253 | ATP2B1 | 0.04485442 |
| NRROS | 0.017323943 | RARS2 | 0.04501746 |
| TEAD1 | 0.017325837 | RP11-292D4.3 | 0.04504629 |
| LINC01442 | 0.017340818 | TTK | 0.04505941 |
| RNF139-AS1 | 0.01735482 | LOC100505501 | 0.04510527 |
| LINC00632 | 0.017359178 | GSN-AS1 | 0.04511589 |
| S100A12 | 0.017373369 | DIS3L | 0.04518604 |
| DQ581328 | 0.017385754 | DQ583756 | 0.0452628 |
| SMAD9 | 0.017412818 | CXCL12 | 0.0453182 |
| KCNJ14 | 0.017438557 | ERICH3-AS1 | 0.04548109 |
| FOXE3 | 0.017447919 | OR1D2 | 0.04554322 |
| GGH | 0.017462922 | NOP16 | 0.04554509 |
| ROS1 | 0.017477578 | AK8 | 0.04555552 |
| GLO1 | 0.017602356 | NEDD1 | 0.04555845 |
| LOC101927438 | 0.017626854 | ZMYND11 | 0.04558961 |
| RPL14 | 0.017640156 | RASSF9 | 0.04562518 |
| IGHA1\|IGHA2\|IGHG1\| | 0.017657629 | TGIF2LY | 0.04563363 |
| IGHG4\|IGHM\|IGHV4- | | | |
| 31\|LOC102723407 | | | |
| TBC1D4 | 0.017668375 | LOC400891 | 0.04572384 |
| LINC00615 | 0.017670542 | XPC | 0.04573976 |
| DEPDC7 | 0.017740941 | CHRNA6 | 0.04579577 |
| PHTF2 | 0.017764821 | ESYT3 | 0.04592974 |
| PPFIA2 | 0.017809634 | OR51B2 | 0.04596962 |
| SULF1 | 0.017953864 | STX11 | 0.04602508 |
| KIAA0355 | 0.017987506 | TMEM38B | 0.04603996 |
| PHKA1 | 0.01801868 | TMEM176B | 0.04607794 |
| UCK1 | 0.018053244 | TMEM257 | 0.04615602 |
| LRCH3 | 0.01808591 | SHC4 | 0.04617231 |
| C20orf26 | 0.018096019 | PGBD5 | 0.04618551 |
| BEX2 | 0.018134016 | MAGIX | 0.0462043 |
| GNL2 | 0.018150297 | RAB2A | 0.04631494 |
| PCDHGA8 | 0.018168144 | TXLNGY | 0.04635109 |
| BC040886\|RP11- | 0.01816878 | LINC00958 | 0.04639737 |
| 804F13.1 | | | |
| SEC14L3 | 0.018209337 | GNL1 | 0.04650732 |
| XRCC6BP1 | 0.018233339 | FAM57A | 0.04657439 |
| KCNK9 | 0.018257919 | CD5L | 0.04658712 |
| LOC102723927 | 0.018414548 | ARHGEF4 | 0.04659686 |
| KCNQ2 | 0.018539864 | LINC00927 | 0.04661223 |
| GAL | 0.018658116 | MYRF | 0.04661628 |
| RP11-218C14.8 | 0.018749159 | C14orf178 | 0.04662153 |
| RP11-295G20.2 | 0.018754835 | RBBP6 | 0.04662491 |
| FAM46C | 0.018833577 | TAPBPL | 0.04664087 |
| LOC101929880\|QPRT | 0.018876841 | AK055981 | 0.04669263 |
| PSMG3 | 0.019002741 | BCAR1 | 0.04669614 |
| CACNB4 | 0.019006215 | ACOT8 | 0.04670456 |
| TRPM5 | 0.019077997 | IFI44L | 0.04676023 |
| SIM2 | 0.019124172 | FAM109B | 0.04694844 |
| C14orf1 | 0.019125694 | CLDN8 | 0.04708612 |
| SAMD10 | 0.019145152 | MAGI2-AS3 | 0.04710411 |
| ATXN7L1 | 0.019205894 | CXCL17 | 0.0471102 |
| GLUL | 0.019242821 | S100A5 | 0.04721846 |
| ITGAV | 0.019246055 | JOSD1 | 0.04737737 |
| LOC101928844 | 0.019286063 | CBR4 | 0.04738291 |
| ERVMER34-1 | 0.019397938 | ITGAD | 0.04741399 |
| DNAJC10 | 0.019415178 | PIEZO1 | 0.04742397 |
| NMUR1 | 0.019512505 | TNFSF14 | 0.04745871 |
| LINC00917 | 0.019539343 | SH2D1A | 0.04749763 |
| PCOLCE | 0.019554613 | HOMEZ | 0.04752578 |
| CACNA2D1 | 0.01957154 | LOC101927499 | 0.04755642 |
| ERV9-1 | 0.019587159 | CD83 | 0.04758177 |
| NPIPA5\|NPIPB3\|NPIPB6\| | 0.019608007 | SHF | 0.04765955 |
| NPIPB8 | | | |
| DBNL\|MIR6837 | 0.01963665 | CBX8 | 0.04770057 |
| SMARCA4 | 0.01969193 | RUNX2 | 0.04779349 |
| QDPR | 0.019712286 | HN1 | 0.04782756 |
| C1orf226 | 0.01989977 | AKR1C2\|LOC101930400 | 0.04784936 |
| ZHX2 | 0.019941186 | CARD11 | 0.04785847 |
| LOC441454 | 0.019960528 | EXD3 | 0.04786946 |
| PRM3 | 0.019990746 | TET1 | 0.04799855 |
| FAM208B | 0.020007798 | KLF13 | 0.0480296 |
| CTB-1202.1 | 0.020022913 | HEATR5A | 0.04812909 |
| KANSL1 | 0.020062867 | ZNF280B | 0.04816551 |
| CHRNE | 0.020095459 | KLHDC1 | 0.0481714 |
| TEX9 | 0.020107954 | ATG4C | 0.04822037 |
| HECW1-IT1 | 0.020232223 | S1PR4 | 0.04828513 |
| PPP1R9B | 0.020247914 | CHAC2 | 0.04828534 |
| ACSF3 | 0.020350163 | IL1RL2 | 0.0483246 |
| PPARGC1B | 0.020448827 | PDCD11 | 0.04836495 |
| ZNF121 | 0.020464406 | INPP4A | 0.04839941 |
| FREM3 | 0.020521961 | LTBP3 | 0.04856128 |
| TNIP1 | 0.020578746 | GTF3C4 | 0.04857095 |
| LOC101928535 | 0.020583019 | ATP5J2-PTCD1\|PTCD1 | 0.04864019 |
| SRPRB | 0.020590861 | IPO4 | 0.04874236 |
| STAT4 | 0.020595322 | RP11-231E19.1 | 0.04895221 |
| RP11-348B17.1 | 0.020615251 | PUS7 | 0.04895761 |
| LOC285692 | 0.020626682 | TGFBI | 0.04896598 |
| LOC100507600 | 0.020636249 | ARL16 | 0.0489804 |
| DHX33 | 0.020760233 | NXT2 | 0.04898659 |
| 6-Sep | 0.020815017 | MBP | 0.04899448 |
| MRPS2 | 0.020815542 | TEX22 | 0.04901049 |
| PHACTR3 | 0.020857105 | SEMA3F | 0.04901428 |
| LOC100131864 | 0.020860209 | FLJ35934 | 0.04902773 |
| BC039537\|RP11- | 0.020870312 | FPR2 | 0.04906107 |
| 30L15.6 | | | |
| FAM53B | 0.020941419 | TNS4 | 0.04911817 |
| LIPA | 0.02098576 | SIVA1 | 0.0491728 |
| RDX | 0.020986973 | RREB1 | 0.04918727 |
| DPH2 | 0.021001424 | C22orf46 | 0.04922849 |
| ZNF518A | 0.021034583 | ARRDC4 | 0.04923372 |
| MEG9 | 0.02112748 | SCARA3 | 0.0492568 |
| TAS2R5 | 0.021145977 | CDK19 | 0.04929166 |
| CRTAP | 0.021314433 | CCDC50 | 0.04933845 |
| ASPSCR1 | 0.021496717 | MORC1 | 0.04935828 |
| CD163 | 0.021502878 | METAP1 | 0.049359 |
| ENOX1 | 0.021559118 | FAM208A | 0.04937109 |
| HK2\|RP11-259N19.1 | 0.02160036 | DNAJC22 | 0.04939061 |
| TRMT10C | 0.021711933 | KRT34\|LOC100653049 | 0.04940886 |
| ITGA4 | 0.022089328 | RAB6B | 0.04952527 |
| RNF212 | 0.022108697 | CYP8B1 | 0.04955061 |
| NIFK | 0.022140204 | SERTAD1 | 0.04969048 |
| FAM69C | 0.022151005 | RP11-326I11.5 | 0.04972402 |
| LOC101929668 | 0.022156268 | BNC2 | 0.04974964 |

TABLE S2

| GCB | | ABC | |
|---|---|---|---|
| gene_id | coefficient | gene_id | coefficient |
| TNFRSF10A | 0.50855087 | CRCP | 0.34501338 |
| CPT1A | 0.4488654 | ZNF518A | 0.34092866 |
| ELOVL6 | 0.41996875 | SLC5A12 | 0.22248288 |
| SNHG4 | 0.32117696 | TMEM37 | 0.19866542 |
| RP11-349E4.1 | 0.24352161 | EPOR\|RGL3 | 0.17316804 |
| HAS3 | 0.17152484 | LINC00917 | 0.17000599 |
| LINC00933 | 0.12532876 | CTB-43E15.1 | 0.16269888 |
| CCDC126 | −0.0021095 | ECT2 | 0.13665093 |
| CALML5 | −0.1004191 | IGSF9 | 0.05431469 |
| CD58 | −0.1764475 | PLCB4 | −0.0738575 |
| LOC339539 | −0.2580686 | LINC00599\|MIR 124-1 | −0.0931187 |
| SERTAD1 | −0.2980318 | ING2 | −0.1009484 |
| | | FAF1 | −0.1500086 |
| | | ZNF236 | −0.1751014 |
| | | AC091633.3 | −0.1898451 |
| | | USH2A | −0.1979775 |

TABLE S3

| GEO number/ | | | Median value |
|---|---|---|---|
| source | Platforms | Use | cutoff¹ |
| GSE10846 | Affymetrix Human Genome | defining gene signature | −8.422649568 |
| | U133 Plus 2.0 Array | for R-CHOP DLBCL | |
| GSE34171 | Affymetrix Human Genome | validate 33 gene signature | −420.221149 |
| | U133 Plus 2.0 Array | | |
| GSE32918/69051 | Illumina HumanRef-8 | validate 33 gene signature | −13.7565591 |
| | WG-DASL v3.0 | | |
| DLBC data from | RNA-Seq | validate 33 gene signature | −1206.356707 |
| TCGA | | | |

¹Calculated reference standard for each sample included in each study/analysis.

TABLE S4

| | | | Calculated Risk |
|---|---|---|---|
| gene_id | coef | GSM275076_Expression | Score |
| ADRA2B | 0.05929974 | 6.327 | 0.37518945 |
| ALDOC | −0.2266974 | 8.693 | −1.9706805 |
| ASIP | −0.0994086 | 2.807 | −0.2790399 |
| ATP8A1 | −0.052468 | 6.644333333 | −0.3486149 |
| CD1E | −0.1111254 | 6.43 | −0.7145363 |
| DUSP16 | −0.0963421 | 6.2495 | −0.60209 |
| ECT2 | 0.13182723 | 3.233 | 0.42619743 |
| ELOVL6 | 0.055146 | 7.19 | 0.39649974 |
| FAF1 | −0.0652772 | 5.905 | −0.3854619 |
| FAM223A\|FAM223B | −0.0121265 | 6.619 | −0.0802653 |
| GAREM | −0.0299263 | 6.363 | −0.190421 |
| GNG8 | −0.0089058 | 5.096 | −0.045384 |
| IGSF9 | 0.19446142 | 2.379 | 0.46262372 |
| LMO2 | −0.0070721 | 2.888 | −0.0204242 |
| LPPR4 | −0.1433395 | 7.409 | −1.0620024 |
| LY75 | −0.252489 | 12.338 | −3.1152093 |
| MAEL | −0.086909 | 5.94 | −0.5162395 |
| NEK3 | 0.08073014 | 9.184 | 0.74142561 |
| PADI2 | −0.0332634 | 6.9495 | −0.231164 |
| PDK1 | −0.0435511 | 7.917 | −0.3447941 |
| PDK4 | 0.18311325 | 5.691333333 | 1.04215854 |
| PES1 | 0.09271489 | 7.3955 | 0.68567297 |
| PPP1R7 | −0.2483229 | 8.731 | −2.1681072 |
| PUSL1 | 0.14247471 | 8.958 | 1.27628845 |
| SCN1A | −0.054923 | 0.766 | −0.042071 |
| SLAMF1 | −0.0094785 | 7.8005 | −0.073937 |
| SSTR2 | −0.0260066 | 5.4075 | −0.1406307 |
| TADA2A | 0.12055065 | 6.901 | 0.83192004 |
| TNFRSF9 | −0.004922 | 7.2755 | −0.03581 |
| USH2A | −0.1920536 | 5.142 | −0.9875396 |
| VEZF1 | −0.3893348 | 10.5915 | −4.1236395 |
| WDR91 | −0.0041198 | 7.185 | −0.0296008 |
| ZMYND19 | 0.26520514 | 8.828 | 2.34123098 |
| | | Total Score | −8.9284561 |

1. A method for diffuse large B-cell lymphoma prognosis and treatment in a patient in need thereof, said method comprising:
determining a first gene expression profile in a biological sample from the patient for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91; and
correlating increased expression levels of said genes with improvement in overall survival outcomes in the patient and administering a therapeutic treatment to said patient.

2. The method of claim 1, further comprising:
determining a second gene expression profile in said biological sample for at least a second set of genes ADRA2B, ECT2, ELOVL6, IGSF9, NEK3, PDK4, PES1, PUSL1, TADA2A, and ZMYND19; and
correlating low expression levels of said second set of genes with improvement in overall survival outcomes in the patient.

3. The method of claim 1, wherein said sample is lymph node tissue.

4. The method of claim 1, wherein said first gene expression profile is determined by detecting the expression level of at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91 in the patient sample.

5. The method of claim 2, wherein said second gene expression profile is determined by detecting the expression level of at least ADRA2B, ECT2, ELOVL6, IGSF9, NEK3, PDK4, PES1, PUSL1, TADA2A, and ZMYND19 in the patient sample.

6. The method of claim 1, wherein said first gene expression profile is determined by a system configured to assay a plurality of molecular targets in the biological sample to detect gene expression levels for said first set of genes, wherein said system is selected from the group consisting of microarray, PCR, immunoassay, quantitative PCR, and next-generation sequencing.

7-8. (canceled)

9. The method of claim 1, further comprising repeating the determination of the first gene expression profile after administering said treatment to yield an updated first gene expression profile, and comparing the first gene expression profile to the updated first gene expression profile to determine efficacy of said treatment.

10-11. (canceled)

12. A method of treating diffuse large B-cell lymphoma in a patient in need thereof, said method comprising:
receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, WDR91, ADRA2B, ECT2, ELOVL6, IGSF9, NEK3, PDK4, PES1, PUSL1, TADA2A, and ZMYND19 detected in a biological sample from the patient;
determining a risk score for said patient based upon increased or decreased expression of each of said gene expression values as compared to a reference standard; and
administering a therapeutic agent to said patient to treat said diffuse large B-cell lymphoma, wherein said therapeutic agent comprises a standard of care active agent when said risk score is low and wherein said therapeutic agent comprises an adjunctive chemotherapeutic, experimental therapy, and/or aggressive active agent against said diffuse large B-cell lymphoma when said risk score is high.

13. The method of claim 12, wherein said standard of care active agent comprises cyclophosphamide, hydroxydaunorubicin, oncovin, prednisone, and anti-CD20 monoclonal antibody rituximab.

14. The method of claim 12, further comprising assessing clinical information regarding said patient, such as tumor size, tumor grade, lymph node status, lymphoma subtype, and family history to evaluate the prognosis of said patient and develop a treatment strategy for said patient.

15. The method of claim 14, wherein said clinical information further includes an IPI or R-IPI risk score.

16. A system for diffuse large B-cell lymphoma prognosis and treatment in a patient in need thereof, said system comprising:
user interface for receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91 in a biological sample from the patient to generate a first gene expression profile;
computer readable memory to store said first gene expression profile;
at least one database comprising a reference standard for each of the first set of genes;
a processor with a computer-readable program code comprising instructions for comparing the first gene expression profile with the reference standard data correlating increased expression levels of said first set of genes with improvement in overall survival outcomes in the patient, and calculating a risk score; and
an output for reporting a risk score for said patient.

17. The system of claim 16, wherein, said user interface is configured for receiving gene expression values for at least ADRA2B, ECT2, ELOVL6, IGSF9, NEK3, PDK4, PES1, PUSL1, TADA2A, and ZMYND19 in said biological sample to generate a second gene expression profile;
computer readable memory to store said second gene expression profile;
at least one database comprising a reference standard for each of the second set of genes; and
a processor with a computer-readable program code comprising instructions for comparing the second gene expression profile with the reference standard data correlating low expression levels of said second set of genes with improvement in overall survival outcomes in the patient and calculating a risk score; and
an output for reporting a risk score for said patient.

18. The system of claim 16, said user interface is configured for receiving an IPI or R-IPI risk score value and an output for comparing said calculated risk score with said IPI or R-IPI risk score.

19. The system of claim 16, wherein said calculation of risk score comprises multiplying each expression value by a reference coefficient value and summing said multiplied value for all expression values to generate said risk score.

20. A method for diffuse large B-cell lymphoma prognosis and treatment in a patient in need thereof, said method comprising:
receiving gene expression values for at least ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, and WDR91 in a biological sample from the patient;
generating a first gene expression profile;
comparing the first gene expression profile with a reference standard data for each of said genes;
correlating increased expression levels of said first set of genes with improvement in overall survival outcomes in the patient; and
calculating a risk score predictive of overall survival for said patient.

21. The method of claim 20, further comprising receiving gene expression values for at least ADRA2B, ECT2, ELOVL6, IGSF9, NEK3, PDK4, PES1, PUSL1, TADA2A, and ZMYND19 in said biological sample from the patient;
generating a second gene expression profile;
comparing the second gene expression profile with a reference standard data for each of said genes;
correlating low expression levels of said second set of genes with improvement in overall survival outcomes in the patient; and
calculating a risk score predictive of overall survival for said patient.

22. The method of claim 20, modifying treatment of said patient based upon said calculated risk score.

23. The method of claim 22, wherein said patient has received treatment for diffuse large B-cell lymphoma prior to detection of said gene expression values.

24-29. (canceled)

30. A kit for diffuse large B-cell lymphoma prognosis and treatment in a patient in need thereof, said kit comprising:
a plurality of probes each having binding specificity for a target gene in a gene panel comprising ALDOC, ASIP, ATP8A1, CD1E, DUSP16, FAF1, FAM223A1FAM223B, GAREM, GNG8, LMO2, LPPR4, LY75, MAEL, PADI2, PDK1, PPP1R7, SCN1A, SLAMF1, SSTR2, TNFRSF9, USH2A, VEZF1, WDR91, ADRA2B, ECT2, ELOVL6, IGSF9, NEK3, PDK4, PES1, PUSL1, TADA2A, and ZMYND19, or a gene product thereof;
optional reagents and/or buffers; and
instructions for mixing said probes with a biological sample obtained from said patient.

31. (canceled)
